SEQUENCE LISTING (46 sequences)

SEQ ID NO: 1 - protein, 20 aa, Homo sapiens
SFRNGVGTGMKKTSFQRAKS

SEQ ID NO: 2 - protein, 20 aa, chimpanzee
SFRNGVGTGMKKTSFRRAKS

SEQ ID NO: 3 - protein, 20 aa, mouse
SFRNGVGSGAKKTSFRRAKQ

SEQ ID NO: 4 - protein, 20 aa, rat
SFRNGVGSGVKKTSFRRAKQ

SEQ ID NO: 5 - protein, 20 aa, dog
SFRNGVGTGMKKTSFRRAKS

SEQ ID NO: 6 - protein, 20 aa, chicken
SFRNGVGSGIKKTSFRRAKS

SEQ ID NO: 7 - protein, 20 aa, Artificial Sequence
  MUTAGEN
SFRNGVGTGMKKTSFQRAKQ

SEQ ID NO: 8 - protein, 20 aa, Artificial Sequence
  MUTAGEN
SFRNGVGTGMKKTSFRRAKQ

SEQ ID NO: 9 - protein, 20 aa, Artificial Sequence
  MUTAGEN
SFRNGVGTGAKKTSFQRAKS

SEQ ID NO: 10 - protein, 20 aa, Artificial Sequence
  MUTAGEN
SFRNGVGTGVKKTSFQRAKS

SEQ ID NO: 11 - protein, 20 aa, Artificial Sequence
  MUTAGEN
SFRNGVGTGIKKTSFQRAKS

SEQ ID NO: 12 - protein, 20 aa, Artificial Sequence
  MUTAGEN
SFRNGVGTGAKKTSFQRAKQ

SEQ ID NO: 13 - protein, 20 aa, Artificial Sequence
  MUTAGEN
SFRNGVGTGVKKTSFQRAKQ

SEQ ID NO: 14 - protein, 20 aa, Artificial Sequence
  MUTAGEN
SFRNGVGTGIKKTSFQRAKQ

SEQ ID NO: 15 - protein, 20 aa, Artificial Sequence
  MUTAGEN
SFRNGVGTGAKKTSFRRAKQ

SEQ ID NO: 16 - protein, 20 aa, Artificial Sequence
  MUTAGEN
SFRNGVGTGVKKTSFRRAKQ

SEQ ID NO: 17 - protein, 20 aa, Artificial Sequence
  MUTAGEN
SFRNGVGTGIKKTSFRRAKQ

SEQ ID NO: 18 - protein, 20 aa, Artificial Sequence
  MUTAGEN
SFRNGVGTGAKKTSFRRAKS

SEQ ID NO: 19 - protein, 20 aa, Artificial Sequence
  MUTAGEN
SFRNGVGTGVKKTSFRRAKS

SEQ ID NO: 20 - protein, 20 aa, Artificial Sequence
  MUTAGEN
SFRNGVGTGIKKTSFRRAKS

SEQ ID NO: 21 - protein, 20 aa, Artificial Sequence
  MUTAGEN
SFRNGVGSGAKKTSFRRAKS

SEQ ID NO: 22 - protein, 20 aa, Artificial Sequence
  MUTAGEN
SFRNGVGSGVKKTSFRRAKS

SEQ ID NO: 23 - protein, 20 aa, Artificial Sequence
  MUTAGEN
SFRNGVGSGMKKTSFRRAKS

SEQ ID NO: 24 - protein, 20 aa, Artificial Sequence
  MUTAGEN
SFRNGVGSGIKKTSFRRAKQ

SEQ ID NO: 25 - protein, 20 aa, Artificial Sequence
  MUTAGEN
SFRNGVGSGMKKTSFRRAKQ

SEQ ID NO: 26 - protein, 20 aa, Artificial Sequence
  MUTAGEN
SFRNGVGSGAKKTSFQRAKQ

SEQ ID NO: 27 - protein, 20 aa, Artificial Sequence
  MUTAGEN
SFRNGVGSGVKKTSFQRAKQ

SEQ ID NO: 28 - protein, 20 aa, Artificial Sequence
  MUTAGEN
SFRNGVGSGIKKTSFQRAKQ

SEQ ID NO: 29 - protein, 20 aa, Artificial Sequence
  MUTAGEN
SFRNGVGSGMKKTSFQRAKQ

SEQ ID NO: 30 - protein, 20 aa, Artificial Sequence
  MUTAGEN
SFRNGVGSGAKKTSFQRAKS

SEQ ID NO: 31 - protein, 20 aa, Artificial Sequence
  MUTAGEN
SFRNGVGSGVKKTSFQRAKS

SEQ ID NO: 32 - protein, 20 aa, Artificial Sequence
  MUTAGEN
SFRNGVGSGIKKTSFQRAKS

SEQ ID NO: 33 - protein, 20 aa, Artificial Sequence
  MUTAGEN
SFRNGVGSGMKKTSFQRAKS

SEQ ID NO: 34 - protein, 20 aa, Artificial Sequence
  MUTAGEN
SFRNGVGLGAKKTSFQRAKS

SEQ ID NO: 35 - protein, 20 aa, Artificial Sequence
  MUTAGEN
SFRNGVGPGVKKTSFQRAKQ

SEQ ID NO: 36 - protein, 20 aa, Artificial Sequence
  MUTAGEN
SFRNGVGWGIKKTSFRRAKS

SEQ ID NO: 37 - protein, 20 aa, Artificial Sequence
  MUTAGEN
SFRNGVGFGMKKTSFRRAKQ

SEQ ID NO: 38 - protein, 20 aa, Artificial Sequence
  MUTAGEN
SFRNGVGLGAKKTSFQRAKS

SEQ ID NO: 39 - protein, 20 aa, Artificial Sequence
  MUTAGEN
SFRNGVGPGVKKTSFQRAKQ

SEQ ID NO: 40 - protein, 20 aa, Artificial Sequence
  MUTAGEN
SFRNGVGWGIKKTSFRRAKS

SEQ ID NO: 41 - protein, 20 aa, Artificial Sequence
  MUTAGEN
SFRNGVGFGMKKTSFRRAKQ

SEQ ID NO: 42 - protein, 20 aa, Artificial Sequence [sequence omitted]

SEQ ID NO: 43 - protein, 7 aa, Homo sapiens
SFRNGVG

SEQ ID NO: 44 - protein, 5 aa, Homo sapiens
KKTSF

SEQ ID NO: 45 - protein, 4 aa, Homo sapiens
RAKS

SEQ ID NO: 46 - protein, 4 aa, Homo sapiens
RAKQ

(19) United States
(12) Patent Application Publication
(10) Pub. No.: US 2016/0279204 A1
(43) Pub. Date: Sep. 29, 2016

(54) PEPTIDES AND PHARMACEUTICAL COMPOSITIONS FOR USE IN THE TREATMENT BY NASAL ADMINISTRATION OF PATIENTS SUFFERING FROM ANXIETY AND SLEEP DISORDERS

(71) Applicant: MAX-PLANCK-GESELLSCHAFT ZUR FÖRDERUNG DER WISSENSCHAFT E.V., MUNICH (DE)

(72) Inventors: Yi-Chun YEN, Munich (DE); Axel STEIGER, Munich (DE); Florian HOLSBOER, Munich (DE); Rainer LANDGRAF, Sinzing (DE); Irina Alexandra IONESCU, Munich (DE); Ulrike SCHMIDT, Munich (DE)

(73) Assignee: MAX-PLANCK-GESELLSCHAFT ZUR FORDERUNG DER WISSENSCHAFT E.V., MUNICH (DE)

(21) Appl. No.: 14/668,939

(22) Filed: Mar. 25, 2015

Related U.S. Application Data
(63) Continuation of application No. 14/009,234, filed on Dec. 4, 2013, now abandoned.
(60) Provisional application No. 61/470,997, filed on Apr. 1, 2011.

(51) Int. Cl. A61K 38/22; A61K 9/00

(52) U.S. Cl. CPC A61K 38/2271; A61K 9/0043

(57) ABSTRACT

The present invention provides peptides for use in a medicament which is administered nasally, wherein the peptide is an agonist of neuropeptide S receptor (NPSR), of the receptor TGR23 and/or of vasopressin receptor-related receptor 1 (VRR1) or for use in the treatment of a patient by causing, promoting or increasing relieve or healing of phobic anxiety, avoidance anxiety, dis-sociative anxiety such as flashbacks, depersonalisation, derealisation, intrusions, vegetative symptoms related to anxiety symptoms, especially in panic attacks, in posttraumatic stress disorder, in generalised anxiety disorder and in anxiety accompanying depressive, or psychotic episodes, arousal, awakening, alertness, activity, spontaneous movement, an anxiolytic effect or a combination thereof in the patient, wherein the peptide is administered nasally or for use in the prophylaxis and/or treatment of an anxiety or sleep dis-order, especially in any type of hypersomnia like idiopathic hypersomnia, wherein the peptide is administered nasally. Further provided are pharmaceutical compositions for nasal administration comprising at least one of said peptides, uses of said peptide or said pharmaceutical composition. The invention also provides a method for identifying target neurons of a peptide in an animal, wherein the peptide is administered nasally.

### CROSS REFERENCE TO RELATED APPLICATIONS

This application is a continuation of U.S. patent application Ser. No. 14/009,234, filed Dec. 4, 2013, which is a U.S. National Stage of PCT/EP2012/056002, filed on Apr. 2, 2012, which claims priority and the benefits of U.S. Patent Application Ser. No. 61/470,997, filed Apr. 1, 2011, each of which is hereby incorporated by reference in the present disclosure in its entirety.

### SUBMISSION OF SEQUENCE LISTING ON ASCII TEST FILE

The content of the following submission on ASCII text file is incorporated herein by reference in its entirety: a computer readable form (CRF) of the Sequence Listing (file name: 717512000201SEQLIST.TXT, date recorded: Mar. 25, 2015, size: 13 KB).

### FIELD OF THE INVENTION

The invention relates to peptides and pharmaceutical compositions for use in the treatment of patients suffering from anxiety and sleep disorders.

### BACKGROUND

Anxiety and sleep disorders affect millions of people. Anxiety disorders comprise inter alia panic disorder, generalized anxiety disorder, phobias and posttraumatic stress disorders. Pathological fear and anxiety can occur in a continuous mode or intermittently. Typical symptoms accompanying pathological fear and anxiety are avoidance behaviour sometimes leading to social isolation, physical ailments like tachycardia, dizziness and sweating, mental apprehension, stress and tension. The strength of these symptoms ranges from nervousness and discomfort to panic and terror in a humans or animals. Most anxiety disorders may last for weeks or even months, some of them even for years and worsen if not treated suitably.

Persistent or intermittent sleep disturbances may accompany other psychiatric or physical disorders or constitute distinct independent disease patterns. Patients suffering from sleep disturbances sleep either too less or too much or their sleep is disturbed by parasomnias like somnambulism. Sleep disturbances or disorders very often lead to a decreased quality of life, diminished concentration and physical illness.

Agonists of neuropeptide S receptor (NPSR) (also known as TGR23 receptor and/or of vasopressin receptor-related receptor 1 (VRR1)) such as neuropeptide S (NPS) have been shown to elicit strong anxiolytic effects in rodents upon intracerebral and intracerebroventricular (ICV) injection (cf. US 2004/0110920; Leonard et al., 2008; Xu et al., 2004). NPS exerts its function via the NPS receptor (NPSR), a G-protein coupled receptor (GPCR) bound to either G_{q} or Gₛ (Reinscheid et al., 2005). NPSR-KO mice are resistant to NPS treatment, showing that NPSR is the only receptor mediating NPS effects (Duangdao et al., 2009). GPCR-internalisation upon ligand binding has not yet been demonstrated for a NPSR-peptide-complex such as the NPSR/NPS-complex.

However, for the development of effective medication for anxiety disorder patients and for medicament approval requirements it is highly important to specifically determine the brain regions which are affected by a particular drug. This also accounts for disorders other than anxiety or sleep disorders.

As to a patient-compliant way of administering NPSR agonists, the most suitable mode of administration for a particular peptide largely depends on the peptide's chemical properties resulting from its specific amino acid sequence and therefore from the chemical nature of the amino acids in the sequence of the peptide, as will be explained in detail below. Each amino acid has distinct chemical properties due to its unique side chain, whereby the amino acids can be regarded as being polar, non-polar, hydrophobic, hydrophilic, basic or acidic. Hence, the specific amino acid composition of a peptide greatly influences its ability to pass the brain-blood-barrier.

Further, in all behavioural studies reported so far which describe the anxiolytic and the arousal-increasing effects of NPS, NPS and other NPSR agonists have been administered to a subject by intracerebral injection techniques. For this purpose, animals have generally been anesthetised with halothane or similar and the NPSR agonist has been injected intracerebroventricularly (ICV) as described in US 2010/0056455, Xu et al. (2004), Laursen and Belknap (1986) or Rizzi et al. (2008). Leonard et al. (2008) described the intracerebroventricular injection of NPS to mice and refer to publications of Malberg et al. (2007) and Ring et al. (2006). Upon intracerebroventricular injection into mice, US 2010/0056455 describes that NPS compositions increased locomotor activity and wakefulness in rodents. However, every single intracerebroventricular administration requires anaesthesia and surgery. Thus, such mode of administration is risky and unpleasant and is contraindicated for patients who require repeated medication administration.

According to US 2004/0110920 NPS compositions are injected directly into the brain or its ventricles.

Further, peptides and proteins are often delivered to a patient by injection, owing to the tendency of these macromolecules to be destroyed by the digestive tract when ingested orally. However, injection therapies have numerous drawbacks such as the discomfort to the patient, poor patient compliance, and the need for administration by trained technicians. Moreover, intravenous or intramuscular injection of substances generally leads to systemic distribution of these substances resulting in systemic side effects.

In summary, there is a need in the art for a patient-compliant way of administration of peptide agonists of the neuropeptide S receptor (NPSR), such as NPS peptides of different origin and mutants and fragments thereof for use in the treatment or prophylaxis of patients suffering from an anxiety or sleep disorder or from a symptom correlated with these disorders. In particular, it is desired to provide a mode of administration which does not suffer from the drawbacks of ICV-injection but is easy to handle by the subject and can be applied without technical assistance.

### SUMMARY OF THE INVENTION

It has been found that intranasally applied NPS enriches in specific target neurons, elicits anxiolytic effects and/or induces distinct changes in the cerebral protein composition. These NPS-induced changes in brain protein composition, as well as the specific accumulation of NPS in its specific target neurons strongly supports the conclusion that besides its anxiolytic properties intranasally administered NPS causes other behavioural effects, like the promotion of alertness and arousal. It has further been found that NPS can be transported from the nasal cavity to brain without losing its biological functions thus identifying the nasal route to be suitable for therapeutic application of NPS and mutants and fragments thereof.

One aspect of the present invention thus concerns a peptide which is an agonist of neuropeptide S receptor (NPSR, also called TGR23 or vasopressin receptor-related receptor 1 (VRR1)) for use in a pharmaceutical composition which is administered nasally. Such a pharmaceutical composition may be used to cause anxiolytic and arousing effects in subjects such as human or animal patients and/or to treat corresponding pathological conditions and related pathological phenomena by nasal administration.

In one embodiment, a peptide for use in the treatment of a subject such as a human or animal patient by causing, promoting or increasing arousal, awakening, alertness, activity, spontaneous movement, an anxiolytic effect, or a combination thereof as well as by relieving or healing avoidance anxiety, dissociative anxiety such as flashbacks, depersonalisation, derealisation and intrusions and vegetative symptoms related to anxiety symptoms, especially in panic attacks, or a combination thereof, may be administered nasally to subjects such as human patients.

In another embodiment, a peptide for use in the prophylaxis and/or treatment of an anxiety or sleep disorder may be administered nasally. In a special embodiment, a peptide for use in the prophylaxis and/or treatment of an anxiety disorder is provided, wherein the peptide is an agonist of neuropeptide S receptor and is administered nasally. The anxiety disorder treated by intranasal application of a peptide of the invention may be selected from the group consisting of panic disorder with and without agoraphobia, phobia, such as animal phobia, social phobia, height anxiety, claustrophobia and agoraphobia, posttraumatic stress disorder, generalised anxiety disorder, any other disease correlated with symptoms of pathological anxiety, and combinations thereof. The sleep disorder treated by intranasal application of a peptide of the invention may be selected from the group consisting of insomnia, hypersomnia, narcolepsy, idiopathic hypersomnia, excessive amounts of sleepiness, lack of alertness, lack of attentiveness, absentmindedness and/or lack of or aversion to movement or exercise, and combinations thereof.

The foregoing embodiments are non-limiting examples of disorders for which the intranasally applied peptides or pharmaceutical formulations of the invention may be used to treat or to prevent.

In another aspect, a method is provided which allows the determination and identification of target neurons and/or target regions of peptides in the brain of an animal, in particular in mammals. This was achieved by tracking the path of intranasally administered fluorescently labelled peptide in the brain. In one embodiment, the peptide may be a neuropeptide such as NPS. By applying this method, brain neuron populations may be specifically stained. Further, target neurons populations identified by this method grossly overlap with the target areas of NPS predicted by detection of NPSR mRNA and protein (Xu et al., 2007; Leonard and Ring, 2011). Using this method, it has further been demonstrated that NPS is internalised by an NPSR-dependent mechanism but not by other internalisation pathways which are likely to cause undesired side-effects and thereby patient discomfort.

### BRIEF DESCRIPTION OF THE DRAWINGS

FIGS. 1A-1D depict a representative selection of mouse brain regions targeted by ICV-administered fluorescent Cy3-NPS. FIG. 1A depicts amygdaloid structures (Cy3-NPS: bright white): central amygdala (CeA), medial amygdala (MeA), basolateral amygdala (BLA), basomedial amygdala (BMA). Cortical structures: dorsal endopiriform cortex (DEn). Basal ganglia: globus pallidus (GP). Scale bar, 200 μm. With reference to FIGS. 1B-1D, leftmost panels show a schematic overview of murine brain regions (Franklin and Paxinos, 2007). With reference again to FIGS. 1B-1D, middle panels show nuclear counterstain DAPI (blue) (scale bar, 100 μm) and cell populations having taken up Cy3-NPS (red). The images in the red channel are presented in two different magnifications (scale bars, 100 μm and 10 μm)—white rectangles indicate area of magnification. With reference again to FIGS. 1B-1D, rightmost panels show an overlay of the blue and red channels (scale bar, 100 μm). FIG. 1B depicts thalamic structures: paraventricular thalamic nucleus (PV), sporadically in medial habenula (MHb), lateral habenula (LHb), mediodorsal thalamic nucleus (MD): medial (MDM), central (MDC) and lateral (MDL). Third ventricle (3V). FIG. 1C depicts hypothalamic structures: periventricular hypothalamic nucleus (Pe), dorsomedial hypothalamic nucleus (DM), ventromedial hypothalamic nucleus (VMH), arcuate hypothalamic nucleus (Arc). Third ventricle (3V). FIG. 1D depicts brainstem structures: central gray of the pons (CGPn), medial vestibular nucleus (MVe), sporadically in posterodorsal tegmental nucleus (PDTg), Barrington's nucleus (Bar), sporadically in locus coeruleus (LC) and in medial parabrachial nucleus (MPB). Fourth ventricle (4V). All images were taken with a confocal microscope and are representative for a total of 10 mice. See Table 2 for a complete list of brain regions where uptake of Cy3-NPS was detected.

FIGS. 2A-2D show the analysis of cell types targeted by Cy3-NPS. Cy3-NPS: red; nuclear counterstain DAPI: blue. FIG. 2A depicts a representative overview image of the hippocampus. Scale bar, 100 μm. FIG. 2B depicts a morphologically representative cells from the granular dentate gyrus. Granular dentate gyrus (GrDG), molecular dentate gyrus (MoDG). Scale bar, 20 μm. Z-stack of 15 images in 0.59 μm intervals. FIG. 2C depicts co-staining with the neuronal marker neurofilament (NF) (green). This representative image was taken from the dentate gyrus. Scale bar, 20 μm. Z-stack of 10 images in 1 μm intervals. FIG. 2D depicts hippocampal CA3 region after co-staining with glial fibrillary acidic protein (GFAP) (green), an astroglial marker. Z-stack of 18 images in 1 μm intervals. Scale bar, 20 μm. With reference to FIGS. 2A-2D, all images were taken with a confocal microscope from brain sections of Bl6 mice and are representative for a total of 10 mice.

FIGS. 3A and 3B depict intracerebral distribution of Cy3-NPS and rhodamine-NPS shown exemplarily in two brain regions 30 min after ICV delivery of substance (leftmost panels: overview images (Franklin and Paxinos, 2007)). With reference to FIGS. 3A and 3B, the left panel of each figure shows the distribution of rhodamine-NPS (images taken with an epifluorescence microscope, representative for a total of 5 mice) and the right panel of each figure shows the distribution of Cy3-NPS (images taken with a confocal microscope). FIG. 3A shows the distribution in the third ventricle (3V). Hypothalamic structures: anterior parvicellular paraventricular hypothalamic nucleus (PaAP), ventral paraventricular hypothalamic nucleus (PaV), dorsolateral and ventromedial suprachiasmatic nucleus (SChDL, SChVM). FIG. 3B shows the distribution in the optical tract (opt). Amygdaloid structures: medial posteroventral and posterodorsal amygdaloid nuclei (MePV, MePD), posteromedial cortical amygdaloid nucleus (PMCo). Scale bars, 100 μm.

FIG. 4A depicts the intracerebral distribution of unconjugated rhodamine shown exemplarily in a region from the olfactory bulb 30 min after ICV (middle panel) or intranasal administration (right panel). Images were taken with an epifluorescence microscope. Image from the same area 30 min after ICV administration of Cy3-NPS (left panel). Image was taken with a confocal microscope. FIG. 4B depicts the ventral and external parts of the anterior olfactory area (AOV, AOE) (overview image (Franklin and Paxinos, 2007). Scale bars, 20 μm.

FIGS. 5A and 5B depict the analysis of the specificity of Cy3-NPS uptake in vivo and in vitro. FIG. 5A depicts coronal sections through mouse brain (overview left panel; Franklin and Paxinos, 2007) with (right panel) and without (middle panel) pre-injection of native NPS at 5 fold concentration 10 min before ICV administration of Cy3-NPS. Posteroventral nucleus of the medial amygdala (MePV), cortical amygdala (ACo). Optic tract (opt). Additional brain regions are depicted in FIGS. 6A-6C for comparison. All images are representative for a total of 4 mice pre-treated with native NPS before ICV administration of Cy3-NPS. FIG. 5B depicts HEK-cells transiently transfected with EGFP-NPSR (green) after 10 min of incubation with Cy3-NPS (red). Nuclear counterstain: DAPI (blue). Merge panel depicts an overlay of all three channels and shows colocalisation of Cy3-NPS and EGFP-NPSR (yellow) in cytoplasmic (arrows) and perinuclear (arrowheads) vesicular structures. All images were taken with a confocal microscope. Scale bars, 20 μm.

FIGS. 6A-6C depict the uptake of Cy3-NPS after pre-injection of native NPS. With reference to FIGS. 6A-6C, the leftmost panels of each figure show overview images of the respective brain regions (Franklin and Paxinos, 2007). FIGS. 6A-6C depict exemplary images of brain areas from murine brains having received pre-injection of native NPS at 5 fold concentration before ICV administration of Cy3-NPS. FIG. 6A depicts exemplary images from the preoptic area comparing uptake of Cy3-NPS before (right panel) and after (middle panel) pre-injection of native NPS. Median preoptic nucleus (MnPO), the vascular organ of the lamina terminalis (VOLT) and the ventromedial preoptic nucleus (VMPO). FIG. 6B depicts thalamic structures (compare FIG. 1B); and FIG. 6C depicts hypothalamic structures (compare FIG. 1C). Third ventricle: 3V. Scale bars, 100 μm. All images were taken with a confocal microscope.

FIGS. 7A-7G depict intracerebral distribution, behavioural and molecular effects of transnasally delivered NPS. FIG. 7A depicts intraneuronal uptake of Cy3-NPS (red) 30 minutes after intranasal delivery shown exemplarily in the hippocampus. DAPI (blue). Left, hippocampal neuron from the oriens layer (CA3 region). Z-stack of 10 images in 1 μm intervals. Right, hippocampal neuron from the pyramidal layer (CA3 region) after NF staining (green). Scale bars, 20 μm. All images were taken with a confocal microscope and are representative for a total of 3 Bl6 mice. FIGS. 7B-7D present data from behavioural testing of Bl6 and HAB mice 4 hrs after intranasal NPS treatment. FIG. 7B depicts graphs generated from elevated plus maze (EPM) testing. Bl6: n=9 (vehicle), n=10 (NPS). HAB: n=10 (vehicle), n=11 (NPS). FIG. 7C depicts graphs generated from dark-light testing. Bl6: n=9 (vehicle), n=10 (NPS). HAB: n=9 (vehicle), n=1 (NPS). FIG. 7D depicts graphs generated from open field testing. Bl6: n=10 for each group. HAB: n=10 (vehicle), n=11 (NPS). Statistical analysis: one-tailed unpaired t-test. FIGS. 7E-7G depict immunoblot analysis of brain region lysates from Bl6 and HAB mice 24 hrs after intranasal NPS treatment. FIG. 7E depicts GluR1, GluR2 and Glt-1 in prefrontal cortex (Pfc) of Bl6 mice; FIG. 7F depicts synapsin in hippocampus (Hc) of Bl6 mice; and FIG. 7G depicts GluR1 and GluR2 in Pfc of HAB mice. Internal expression control: GAPDH. Blot excerpts show three representative adjacent bands of each group. These data represent cumulated data from at least three independent experiments. Bl6: n=5 for each group. HAB: n=6 for each group. Statistical analysis: two-tailed unpaired t-test. * p<0.05; ** p<0.01. All data are shown ±s.e.m.

FIGS. 8A-8C depict effects of intranasally administered native NPS on behaviour 30 min after treatment in Bl6 and HAB mice. FIG. 8A depicts graphs generated from elevated plus maze (EPM) testing. In Bl6 mice, n=9 (vehicle), n=10 (NPS). In HAB mice, n=10 (vehicle), n=1 (NPS). FIG. 8B depicts graphs generated from dark-light testing. In Bl6 mice, n=9 (vehicle), n=10 (NPS). In HAB mice, n=9 (vehicle), n=1 (NPS). FIG. 8C depicts graphs generated from open field testing. In Bl6 mice, n=10 for each group. In HAB mice, n=9 (vehicle), n=1 (NPS). For Bl6 mice, cumulated data from two experiments are presented. Statistical analysis was performed using the one-tailed unpaired t-test. * p<0.05; ** p<0.01. All data are shown ±s.e.m.

FIGS. 9A-9D depicts additional effects of intranasally administered NPS on protein expression levels in He and Pfc of Bl6 and HAB mice 24 hrs after treatment. FIG. 9A depicts levels of GluR1, GluR2 and Glt-1 in He of Bl6 mice. FIG. 9B depicts levels of synapsin in Pfc of Bl6 mice. FIG. 9C depicts levels of Glt-1 and synapsin in Pfc of HAB mice. FIG. 9D depicts levels of GluR1, GluR2, Glt-1 and synapsin in He of HAB mice. Internal expression control: GAPDH. Blot excerpts show three representative adjacent bands of each group. The data represent cumulated data from at least two independent experiments. Bl6 mice: n=5 for each group. HAB mice: n=6 for each group. Statistical analysis was performed using the two-tailed unpaired t-test. * p<0.05; ** p<0.01. All data are shown ±s.e.m.

FIGS. 10A and 10B depict that microinjections of NPS into the VH reduce anxiety in mice. FIG. 10A depicts that Cy3-NPS is locally restricted to the site of injection into area CA1 of the VH. (Upper panel) Injection site on an anatomical plate (Franklin and Paxinos, 2007). Overlay of DAPI (nuclear staining, blue) and Cy3-NPS (red) signals. Arrow indicates the injection site in the brain section. (Lower panel) Anatomical plate showing the lateral (LA) and basolateral (BLA) amygdala, and overview of the amygdala in a brain section after Cy3-NPS injection (inset: Cy3 channel only). N=4. Scale bars, 200 and 20 μm. FIG. 10B depicts that NPS injections into area CA1 of the VH produce an anxiolytic, locomotion-independent effect on the EPM. (Upper left panel) Anatomical plate showing the injection sites (n=8 mice for each group). (Upper right panel) The distance traveled in the open field is not changed by NPS injection. (Middle panels) Anxiety- and locomotion-related behaviour in the dark-light test is not altered by NPS injection. (Lower panels) NPS injections decreased anxiety-related behaviour on the EPM without affecting locomotion.

FIGS. 11A-11C show that VSDI reveals NPS to weaken evoked neuronal activity flow from the dentate gyrus to area CA1. FIG. 11A provides an illustration of the position of the stimulation electrode (Stim) and the three ROIs used for the calculation of neuronal population activity within the dentate hilus, the CA3 subfield, and area CA1 (Upper panel); and depicts representative filmstrips depicting the propagation of VSDI signals from the dentate gyrus to the CA1 region before (‘Baseline’) and after bath application of 1 μM NPS (‘NPS’). Warmer colours represent stronger neuronal activity. Time specifications are given relative to the electrical stimulation pulse. FIG. 11B illustrates time course of the experiments depicted for the CA1 output subfield of the VH. NPS (1 μM) decreased FDS peak amplitudes (n=7 slices from 6 mice). This effect was completely abolished by a pretreatment (15 min) of slices with the specific NPSR antagonist (R)-SHA 68 (10 μM) (n=7 slices from 4 mice). VSDI recordings were conducted at intervals of 5 min. Data were normalised to the mean FDS peak amplitude of the last two acquisitions during baseline recording. FIG. 11C illustrates quantification Quantification of NPS effects on FDS peak amplitudes in the dentate hilus, the CA3 region, and area CA1. Statistical evaluation was performed by comparing the mean FDS peak amplitudes of the last two acquisitions during baseline recording with the mean FDS peak amplitudes of the last two acquisitions during application of NPS.

FIGS. 12A-12C illustrate that intranasally applied NPS impacts on basal neurotransmission and plasticity at CA3-CA1 synapses of the VH in C57BL/6N mice. FIG. 12A illustrates that intranasal NPS administration caused a shift of the input-output curve towards bigger fEPSP amplitudes (open squares: n=14 slices from 5 mice; closed squares: n=9 slices from 4 mice). FIG. 12B illustrates that intranasally applied NPS reduced paired-pulse facilitation at interstimulus intervals of 25, 50, 100, and 200 ms (open squares: n=14 slices from 5 mice; closed squares: n=11 slices from 4 mice). FIG. 12C illustrates that intranasal NPS application decreased the magnitude of LTP at CA3-CA1 synapses induced by high-frequency stimulation (HFS) (open squares: n=10 slices from 5 mice; closed squares: n=10 slices from 4 mice).

FIGS. 13A-13C illustrate that intranasally applied NPS leads to the same functional alterations at CA3-CA1 synapses in HAB mice as in C57BL/6N mice. FIG. 13A illustrates that intranasal NPS administration caused a shift of the input-output curve towards bigger fEPSP amplitudes (open squares: n=9 slices from 5 mice; closed squares: n=11 slices from 5 mice). FIG. 13B illustrates that intranasally applied NPS reduced paired-pulse facilitation at interstimulus intervals of 25, 50, 100, and 200 ms (open squares: n=9 slices from 5 mice; closed squares: n=12 slices from 5 mice). FIG. 13C illustrates that intranasal NPS application decreased the magnitude of LTP at CA3-CA1 synapses (open squares: n=8 slices from 5 mice; closed squares: n=9 slices from 5 mice).

### DETAILED DESCRIPTION

“Suitable for use by nasal administration” in the sense of the invention means that the peptide is stably applicable to the nose of a human or animal subject and is able to pass the nasal mucosa, i.e. having nasal mucosal permeability, and to reach the intracerebral receptors and/or to cause, promote or increase arousal, awakening, alertness, activity, spontaneous movement, anxiolytic effects or a combination thereof in the subject as well as to relieve or heal avoidance anxiety, dissociative anxiety such as flashbacks, depersonalisation, derealisation and intrusions, vegetative symptoms related to anxiety symptoms, especially in panic attacks, or a combination thereof in the subject, and/or is effective in the prophylaxis and/or treatment of an anxiety or sleep disorder, subsequently. All aforementioned modes of behaviour are to be understood according to their general meaning and in particular according to their meaning in behavioural studies of humans and animals.

“Effective” denotes that the respective effect is achieved.

“Anxiety disorders” may comprise inter alia panic disorder with and without agoraphobia, phobia, such as animal phobia, social phobia, height anxiety, claustrophobia and agoraphobia, posttraumatic stress disorder, generalised anxiety disorder, anxiety symptoms going along with depressive or psychotic episode, any other disease correlated with symptoms of pathological anxiety, and combinations thereof. Anxiety disorders may further comprise pathological fear and anxiety which can occur in a continuous mode or intermittently. Typical symptoms accompanying pathological fear and anxiety are avoidance behaviour sometimes leading to social isolation, stress, tension, physical symptoms and dissociative anxiety, physical ailments like tachycardia, dizziness and sweating, mental apprehension, stress and tension. The strength of these symptoms ranges from nervousness and discomfort to panic and terror in a human or animal.

“Sleep disorders” are usually characterised by symptoms such as an unusual sleep pattern or sleeping behaviour, often ascribed to a neuronal malfunction and/or an dysbalance of the neurotransmitter system which is involved in sleep regulation. Typical examples of sleep disorders are insomnia, hypersomnia, narcolepsy, idiopathic hypersomnia, lack of alertness, lack of attentiveness, absentmindedness and/or lack of or aversion to movement or exercise, excessive amounts of sleepiness, sleep-related breathing disorders, circadian rhythm disorders, parasomnia and sleep related movement disorders and combinations thereof.

In principal, any disorder or disease which is correlated with a low NPS level in the brain or relevant brain regions or with a low NPSR activity level or which is otherwise compensable by elevation of intracerebral NPS levels may be treated with the peptides or pharmaceutical formulations of the present invention.

A “symptom” in the sense of the invention may be any symptom correlated with an anxiety or sleep disorder and is known to s person skilled in the art. Examples of symptoms in anxiety disorders are abnormal fear and pathological fear, anxiety, fearfulness, uncertainty, mental apprehension, stress, tension, vegetative and physical symptoms (i.e. elevation of heart rate and blood pressure, dizziness, sweating, nausea and other symptoms caused by overdrive of the sympathetic nervous system), dissociative anxiety (e.g. flashbacks, intrusions, depersonalization, derealisation) and anxiety related avoidance behaviour each of them in different grades ranging from nervousness and discomfort to panic and terror or a combination thereof. Examples of symptoms in sleep disorders are sleepiness, excessive daytime sleepiness, lack of alertness, lack of attentiveness, absentmindedness and/or lack of or aversion to movement or exercise as well as decreased or diminished arousal, decreased or diminished arousal awakening, decreased or diminished arousal alertness, decreased or diminished arousal activity and decreased or diminished arousal spontaneous movements, each of them in different grades ranging from nervousness and discomfort to panic and terror, or a combination thereof.

The “activity” of a peptide of the invention is understood to mean the property of the peptide to bind to and functionally activate its receptor. In one embodiment, the peptide for use is internalised with the receptor in a receptor-peptide-complex. In general, agonists and antagonists both bind to their respective receptor, the agonist leading to a positive activating response of the receptor and the antagonist leading to a negative response of the receptor and thereby blocking the further signalling pathway. The binding capacity of agonists and antagonist is characterised by the dissociation constant K_{d}. The dissociation constant of NPRS agonists can be determined as described in Xu et al. (2004). Peptide which are agonists of NPRS may have a K_{d} value of 1 μM or lower, optionally of 0.5 μM or lower, of 0.25 μM or lower, of 0.1 μM or lower, of 50 nM or lower, of 25 nM or lower, of 10 nM or lower, of 7 nM or lower, of 5 nM or lower, of 4 nM or lower, or of 2 nM or lower. General principles of agonist and antagonist activity in the field of signal transduction and signalling pathways are known to a person skilled in the art and can be taken from general literature such as Alberts et al. “Molecular Biology of the Cell” (2007, 5. edition, Taylor & Francis, London, UK). The neuropeptide S receptor (NPSR) is also known as TGR23 receptor or vasopressin receptor-related receptor 1 (VRR1) and has been inter alia described in US 2010/0056455 and US 2004/0110920. Thus, NPSR, TGR23 and VRR1, which are understood as synonyms, are all “receptors” in the sense of the invention.

A “peptide” or “polypeptide” is a protein fragment comprising a short chain of amino acids, i.e. a short amino acid sequence, no less than two amino acids. Also explicitly included are peptides or polypeptides having the reverse sequence of any sequence mentioned herein or incorporated by reference. A “protein” is in general a longer chain of amino acids, i.e. amino acid sequence, though there is no exact rule as to where a peptide ends and a protein begins. A peptide can be naturally occurring or be non-naturally occurring. A naturally occurring peptide may be present in nature, e.g. in human, animals, plants or microorganisms such as bacteria or archaea or else. A “non-naturally occurring peptide” is a peptide which does not exist in nature. It may contain conservative and/or non-conservative substitutions, additions and/or deletions of one or more amino acids by any other of the standard amino acids and/or by any other non-standard amino acid.

A non-naturally occurring peptide may be a mutant of a naturally occurring peptide. A “mutant” as used herein denotes a peptide or protein, wherein one or more amino acids are exchanged or substituted by any other of the standard amino acids mentioned herein or by any other non-standard amino acid and/or deleted and/or one or more standard and/or non-standard amino acids are added to while maintaining the activity of the peptide. In case of a conservative amino acid substitution, an amino acid is exchanged under consideration of its chemical nature, e.g. a polar and/or hydrophobic amino acid is only exchanged by another polar and/or hydrophobic amino acid, a non-polar and/or hydrophilic amino acid is only exchanged by another non-polar and/or hydrophilic amino acid, a basic amino acid is only exchanged by another basic amino acid or an acidic amino acid is only exchanged by another acidic amino acid. In one embodiment, there is no limitation whether the substitution must be by a standard or a non-standard amino acid, as long as the chemical nature is maintained. In case of a non-conservative substitution, the chemical nature of the substituted amino acid is not identical to that of the replacing amino acid, e.g. a basic amino acid is not substituted by another basic amino acid but e.g. by an acidic amino acid. Truncated peptides or proteins are also mutants in the sense of the invention. In comparison to natural occurring peptides, mutant peptides may have improved properties such as an increased protease resistance or an improved resistance to chemical degradation, such as methionine oxidation or intrinsic fluorescence. A “fragment” in the sense of the invention is a truncated peptide of the invention, in which e.g. one, two, three, four, five or more amino acid residues are deleted while maintaining the activity of the peptide.

A peptide or protein mutant of the invention has in general at least 70% or 75%, optionally at least 80%, or at least 85%, or even at least 90% or at least 95% identity on the amino acid level to an amino acid sequence given elsewhere in the description or as given in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41 or 42. Usually, the homology is determined over the whole sequence length of the peptides. The same definition applies analogously to a nucleic acid sequence. In the scope of the present invention, the term “identical” is used in reference to amino acid sequences or nucleic acid sequences, meaning that they share a certain degree of “identity”, i.e. “homology” or “similarity”, with another amino acid sequence or nucleic acid sequence, respectively.

Many algorithms exist to determine this degree of identity, homology or similarity. Usually, the homology can be determined by means of the Lasergene software of the company DNA star Inc., Madison, Wis. (USA), using the CLUSTAL method (Higgins et al., 1989, Comput. Appl. Biosci., 5 (2), 151). Other programs that a skilled person can use for the comparison of sequences and that are based on algorithms are, e.g., the algorithms of Needleman and Wunsch or Smith and Waterman. Further useful programs are the Pile Aupa program (J. Mol. Evolution. (1987), 25, 351-360; Higgins et al., (1989), Cabgos, 5, 151-153) or the Gap and Best Fit program (Needleman and Wunsch, (1970), J. Mol. Biol, 48, 443-453, as well as Smith and Waterman (1981), Adv., Appl. Math., 2, 482-489) or the programs of the GCG software package of the Genetics Computer Group (575 Science Drive, Madison, Wis., USA 53711). Sequence alignments can also be performed with the ClustalW program from the internet page http://www.ebi.ac.uk/clustalw or with the NCBI Blast Sequence alignment program from the internet page www.ncbi.nlm.nih.gov/BLAST/ or www.ncbi.nlm.nih.gov/blast/bl2seq/wblast2.cgi.

Also, the skilled person is aware of the techniques which allow him to isolate homologous sequences from other organisms. He can perform homology comparisons (via CLUSTAL, BLAST, NCBI) and then isolate the identified homologous nucleotide or amino acid sequences by means of standard laboratory methods, e.g. primer design, PCR, hybridisation or screening of cDNA libraries with adequate probes (cf. e.g. Sambrook and Russell (2001) Molecular Cloning: A Laboratory Manual, 3. edition, Cold Spring Harbour Laboratory Press, Cold Spring Harbour, N.Y., USA). The function of the identified proteins can then be determined by the method described herein.

An “amino acid” or “amino acid residue” in the sense of the invention contains an amine group, a carboxylic acid group and a side chain which differs from one amino acid to the other, wherein the amine group and the carboxylic group, respectively, form a peptide or amide bond with the preceding or subsequent amino acid residue within the peptide chain. The term “amino acid” refers to standard and non-standard amino acids. 22 standard amino acids are known to date from which only 20 occur in general in human and in animal. These “standard amino acids” and their general abbreviations as three-letter and as one-letter code are summarised in Table 1:

TABLE 1
Standard amino acids and their abbreviations

| Amino Acid | Three-letter | One-letter |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Selenocysteine | Sec | U |
| Pyrrolysine | Pyr | O |

“Non-standard amino acids” or “non-standard amino acid residues” of the present invention are analogues of the standard amino acids in that they are derived from a standard amino acid by chemical variation of the side chain of a standard amino acid. Typically, non-standard amino acids do not participate in protein translation at the ribosome of a cell in nature. However, they may appear in nature and participate in other physiological processes. Also non-standard amino acids contain an amine group, a carboxylic acid group, but differ in their side chain from the standard amino acids as listed in Table 1.

Non-standard amino acids encompass a variety of substances and examples for non-standard amino acids include but are not limited to molecules selected from the group consisting of O-methyl-L-tyrosine, L-3-(2-naphthyl)alanine, 3-methyl-phenylalanine, O-4-allyl-L-tyrosine, 4-propyl-L-tyrosine, tri-O-acetyl-GlcNAcβ-serine, an L-Dopa, a fluorinated phenylalanine, isopropyl-L-phenylalanine, p-azido-L-phenylalanine, p-acyl-L-phenylalanine, p-benzoyl-L-phenylalanine, an L-phospho-serine, a phosphonoserine, a phosphonotyrosine, p-iodo-phenylalanine, homopropargylglycine, azidohomoalanine, p-bromophenylalanine, p-amino-L-phenylalanine and isopropyl-L-phenylalanine. Additionally, other examples of non-standard amino acids optionally include but are not limited to an unnatural analogue of a tyrosine amino acid; an unnatural analogue of a glutamine amino acid, an unnatural analogue of a phenylalanine amino acid, an unnatural analogue of a serine, an unnatural analogue of a threonine, an unnatural analogue of an arginine analogue, an unnatural analogue of an asparagine, an unnatural analogue of a glycine, an unnatural analogue of a valine, an unnatural analogue of a methionine, an unnatural analogue of a lysine, an unnatural analogue of a glutamine, an alkyl, aryl, acyl, azido, cyano, halo, hydrazine, hydrazide, hydroxyl, alkenyl, alkynl, ether, thiol, sulfonyl, seleno, ester, thio-acid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labelled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged amino acid; a photoisomerizable amino acid; a biotin or biotin-analogue containing amino acid; a glycosylated or carbohydrate modified amino acid; a keto containing amino acid; an amino acid comprising polyethylene glycol; an amino acid comprising polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α-disubstituted amino acid; a β-amino acid; and a cyclic amino acid other than proline. Further examples and more information can be taken for example from “Engineering the genetic code” by Budisa (2005, Wiley-VCH, Weinheim, Germany) or from US 2011/027867.

“Unnatural” with respect to amino acids denotes an amino acid which does not naturally occur in proteins or peptides. Standard and non-standard amino acids may be obtained for example from Bachem (Bubendorf, Switzerland), Sigma Aldrich (St. Louis, Mo., USA), AnaSpec (Fremont, Calif., USA) or Alfa Aesar (Ward Hill, Mass., USA).

All amino acids may be grouped according to their chemical properties such as hydrophobicity (non-polar), hydrophilicity (polar), basicity and/or acidity.

In general and also in the sense of the invention, the standard amino acids alanine (Ala, A), valine (Val, V), methionine (Met, M), leucine (Leu, L), isoleucine (Ile, I), proline (Pro, P), tryptophan (Trp, W) and phenylalanine (Phe, F) are regarded as being non-polar and/or hydrophobic and are abbreviated herein as “Φ” or “Φxx”. Thus, “Φ” or “Φ” denote a non-polar and/or hydrophobic amino acid and may optionally be any amino acid selected from the group consisting of alanine (Ala, A), valine (Val, V), methionine (Met, M), leucine (Leu, L), isoleucine (Ile, I), proline (Pro, P), tryptophan (Trp, W) and phenylalanine (Phe, F).

In general and also in the sense of the invention, the amino acids tyrosine (Tyr, Y), threonine (Thr, T), glutamine (Gln, Q), glycine (Gly, G), serine (Ser, S), cysteine (Cys, C) and asparagine (Asn, N) are regarded as being polar and/or neutral and are abbreviated herein as “Ψ” or “Ψxx”. Thus, “Ψ” or “Ψxx” denote a polar and/or neutral amino acid and may optionally be any amino acid selected from the group consisting of tyrosine (Tyr, Y), threonine (Thr, T), glutamine (Gln, Q), glycine (Gly, G), serine (Ser, S), cysteine (Cys, C) and asparagines (Asn, N).

In general and also in the sense of the invention, the amino acids lysine (Lys, K), arginine (Arg, R) and histidine (His, H) are regarded as being basic amino acids and are abbreviated herein as “Ω” or “Ωxx”. Thus, “Ω” or “Ωxx” denote a basic amino acid and may optionally be any amino acid selected from the group consisting of lysine (Lys, K), arginine (Arg, R) and histidine (His, H).

In general and also in the sense of the invention, the amino acids glutamic acid (Glu, E) and aspartic acid (Asp, D) are regarded as being acidic amino acids and are abbreviated herein as “Θ” or “Θxx”. Thus, “Θ” or “Θxx” denote an acidic amino acid and may optionally be any amino acid selected from the group consisting of acids glutamic acid (Glu, E) and aspartic acid (Asp, D).

Further information regarding amino acids and their chemical nature can be taken for example from Hughes (edt.) “Amino Acids, Peptides and Proteins in Organic Chemistry” (2009, Wiley-VCH, Weinheim, Germany) or Jones “Amino acid and peptide synthesis” (2002, Oxford University Press).

In case of non-standard amino acids, the amino acids are in general grouped according to their standard counterpart, e.g. an alanine analogue would be regarded as being a non-polar and/or hydrophobic amino acid or an arginine analogue would be regarded as being a basic amino acid. However, the exact properties of an amino acid depend on its side chain and thus a standard neutral amino acid may have an analogue which is acidic or basic due to a basic or acidic chemical modification of the side chain. In this case, the chemical properties of a non-standard amino acid with respect to its hydrophobicity (non-polar), hydrophilicity (polar), basicity and/or acidity are to be assigned according to standard chemical knowledge and the understanding of a person skilled in the art. Further information can also be taken for example from “Engineering the genetic code” by Budisa (2005, Wiley-VCH, Weinheim, Germany).

In one embodiment of the present invention, a peptide which is an agonist of NPSR and is for use in a medicament or pharmaceutical composition which is administered nasally and is e.g. for use in the treatment of a patient by causing, promoting or increasing arousal, awakening, alertness, activity, spontaneous movement, anxiolytic effects or a combination thereof as well as relieving or healing of avoidance anxiety, dissociative anxiety such as flashbacks, depersonalisation, derealisation and intrusions, vegetative symptoms related to anxiety symptoms, especially in panic attacks, in the patient and wherein the peptide is administered nasally and/or for use in the prophylaxis and/or treatment of an anxiety or sleep disorder, wherein the peptide is administered nasally, comprises the amino acid sequence Z¹ₘZ²ₙSΨΦΩΨΨΦΨΨᵢΨΦⱼΩΩΨΨΦ(Ψₖ or Ωₖ)ΩΦΩ(Ψₗ or Ωₗ)Z²ₚZ³_{q} or Z¹ₘZ²ₙSΨΦΩΨΨΦΨΨᵢΨΦⱼΩΩΨΨΦΨₖΩΦΩΨₗZ²ₚZ³_{q} or Z¹ₘZ²ₙSΨΦΩΨΨΦΨΨᵢΨΦⱼΩΩΨΨΦΩₖΩΦΩΩₗZ²ₚZ³_{q} or Z¹ₘZ²ₙSΨΦΩΨΨΦΨΨᵢΨΦⱼΩΩΨΨΦΨₖΩΦΩΩₗZ²ₚZ³_{q} or Z¹ₘZ²ₙSΨΦΩΨΨΦΨΨᵢΨΦⱼΩΩΨΨΦΩₖΩΦΩΨₗZ²ₚZ³_{q}, and e.g. the amino acid sequence Z¹ₘZ²ₙSSFRNGVGΨᵢGΦⱼKKTSF(Ψₖ or Ωₖ)RAK(Ψₗ or Ωₗ)Z²ₚZ³_{q} or Z¹ₘZ²ₙSSFRNGVGΨᵢGΦⱼKKTSFΨₖRAKΨₗZ²ₚZ³_{q} or Z¹ₘZ²ₙSSFRNGVGΨᵢGΦⱼKKTSFΩₖRAKΩₗZ²ₚZ³_{q} or Z¹ₘZ²ₙSSFRNGVGΨᵢGΦⱼKKTSFΨₖRAKΩₗZ²ₚZ³_{q} or Z¹ₘZ²ₙSSFRNGVGΨᵢGΦⱼKKTSFΩₖRAKΨₗZ²ₚZ³_{q},

wherein Z¹ is an N-terminal blocking group or —NH₂;
Z² is a member selected from the group consisting of one or more basic amino acids such as lysine, arginine and/or histidine, a non-standard amino acid, a fluorescence tag, hydrophobic tag or hydrophilic tag;
Z³ is a C-terminal blocking group or —COOH; and i, j, k, l, m, n, p and q are integers independently selected from 0 to 25; and wherein Φ is a non-polar and/or hydrophobic amino acid or a non-polar and/or hydrophobic non-standard amino acid; wherein Ψ is a is a polar and/or neutral amino acid or a polar and/or neutral non-standard amino acid; wherein Ω is a basic amino acid or a basic non-standard amino acid. All other amino acid abbreviations correspond to the standard abbreviation of amino acids as shown in Table 1.

Non-limiting examples of an N-terminal blocking group are an N-acetyl amino acid, a glycosylated amino acid, a pyrrolidone carboxylate group, an acetylated amino acid, a formylated amino acid, myristic acid, a pyroglutamate conjugated amino acid or else.

Non-limiting examples of a C-terminal blocking group are an amidated amino acid. Other N- or C-terminal blocking groups are known to a person skilled in the art and can also be taken from “Amino acids, peptides, and proteins” by Davies (2006, Royal Society of Chemistry, London, UK), “Biochemistry” by Garrett and Grisham (2010, Cengage Learning, Andover, UK) or from WO 97/39031.

A “hydrophobic tag” can be an amino acid sequence of 1 to 10 amino acids which contains exclusively hydrophobic and/or non-polar amino acids. A “hydrophilic tag” may be an amino acid sequence of 1 to 10 amino acids which contains exclusively hydrophilic and/or polar amino acids.

Also provided is a peptide for use according to the invention, wherein the peptide is a non-naturally occurring peptide and contains conservative and/or non-conservative substitutions, additions and/or deletions.

In another embodiment, the peptide for use of the invention comprises the amino acid sequence Z¹ₘZ²ₙSFRNGVGX¹ᵢGX²ⱼKKTSFX³ₖRAKX⁴ₗZ²ₚZ³_{q}, wherein X¹ is a polar and/or neutral amino acid or a polar and/or neutral non-standard amino acid, optionally a member selected from the group consisting of tyrosine, threonine, glutamine, glycine, serine, cysteine and asparagine; X² is a non-polar and/or hydrophobic amino acid or a non-polar and/or hydrophobic non-standard amino acid, optionally a member selected from the group consisting of alanine, valine, methionine, leucine, isoleucine, proline, tryptophan and phenylalanine; X³ is a polar and/or neutral amino acid or a basic amino acid or a polar and/or neutral non-standard amino acid or a basic non-standard amino acid, optionally a member selected from the group consisting of tyrosine, threonine, glutamine, glycine, serine, cysteine, asparagine, lysine, arginine and histidine; X⁴ is a polar and/or neutral amino acid or a basic amino acid or a polar and/or neutral non-standard amino acid or a basic non-standard amino acid, optionally a member selected from the group consisting of tyrosine, threonine, glutamine, glycine, serine, cysteine, asparagine, lysine, arginine and histidine; Z¹ is an N-terminal blocking group or —NH₂; Z² is a member selected from the group consisting of one or more basic amino acids such as lysine, arginine and/or histidine, a non-standard amino acid, a fluorescence tag, hydrophobic tag or hydrophilic tag; Z³ is a C-terminal blocking group or —COOH; i, j, k, l, m, n, p and q are integers independently selected from 0 to 25.

In another embodiment, the peptide for use according to the invention may comprise the amino acid sequence Z¹ₘZ²ₙSFRNGVG(Tᵢ or Sᵢ)G(Mⱼ or Aⱼ or Vⱼ or Iⱼ)KKTSF(Qₖ or Rₖ)RAK(Sₗ or Qₗ)Z²ₚZ³_{q}, wherein Z¹ is an N-terminal blocking group or —NH₂; Z² is a member selected from the group consisting of one or more basic amino acids such as lysine, arginine and/or histidine, a non-standard amino acid, a fluorescence tag, hydrophobic tag or hydrophilic tag; Z³ is a C-terminal blocking group or —COOH, and i, j, k, l, m, n, p and q are integers independently selected from 0 to 25.

In another embodiment, the peptide comprises an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45 and 46 or a mutant or fragment thereof. Optionally, said sequences further comprise a member selected from the group consisting of an amino acid tag, an amino acid modification, a C-terminal blocking group and/or an N-terminal blocking group, one or more additional basic amino acids such as lysine, arginine and histidine, one or more standard or non-standard amino acids, a fluorescence tag, hydrophobic tag and a hydrophilic tag.

All peptides of the invention may additionally comprise an amino acid tag and/or an amino acid modification.

An “amino acid tag” may consist of one or more standard or non-standard amino acids and may optionally be suitable for purification purposes, e.g. His-tag, a glutathione-S-transferase (GST)-tag, maltose binding protein (MBP)-tag, or is a fluorescence tag such as a member of the cyanine family, e.g. Cy3, rhodamine or a rhodamine derivative, a member of the GFP-family such as green fluorescent protein (GFP), enhanced green fluorescent protein (EGFP), cyan fluorescent protein (CFP), enhanced cyan fluorescent protein (ECFP), yellow fluorescent protein (YFP), enhanced yellow fluorescent protein (EYFP), DsRed, an azatryptophan, or similar, a dye or radioactive label for visual or radioactive detection, an antibody for targeted delivery of the peptide upon administration to the patient or for peptide purification or an antigen for antibody detection. Information on fluorescent proteins and fluorescent tags can further be taken from Sullivan “Fluorescent proteins” (2008, Academic Press, Elsevier, London, UK) and Miller “Probes and tags to study biomolecular function” (2008, Wiley-VCH, Weinheim, Germany).

An “amino acid modification” may be any chemical or biological modification of a standard or non-standard amino acid ranging from simple chemical or biological variations, such as atomic additions, deletions or substitutions to complex chemical modifications, such as a modification corresponding to a posttranslational modification, e.g. the addition of one or more carbohydrates (e.g. mono-, oligo- or multimers), sugar linkers or glycosidic side chains, amino acid phosphorylation, methylation, acetylation, amidation, hydroxylation, sulfation, flavin binding, oxidation and nitrosylation or the chemical addition of other molecules.

Amino acid tags are in general added or linked to the peptide via the N- or C-terminal group, i.e. via —NH₂ or —COOH, whereas amino acid modifications are usually added or linked to the peptide via one or more side chains of the amino acids of the peptide irrespective of whether the amino acid is at or close to the N- or C-terminal end (exo-position) of the peptide or is located at an inner position (endo-positions) of the sequence. Amino acid and peptide modifications and tags are known to a person skilled in the art and additional information can be taken for example from “Posttranslational modification of proteins” by Walsh (2006, Roberts and Company Publishers, Greenwood Village, Colo., USA), “Peptides: chemistry and biology” by Sewald and Jakubke (2009, Wiley-VCH, Weinheim, Germany) and “Peptide and Protein Design for Biopharmaceutical Applications” by Jensen (2009, John Wiley and Sons, Hoboken, N.J., USA).

A “fluorescent amino acid” is a standard or non-standard amino acid being intrinsically fluorescent, such as tryptophan, tyrosine, phenylalanine or their analogues such as aza- or hydroxyltryptophans and else. Fluorescent amino acids are known to a person skilled in the art and further information can be taken for example from Hughes (edt.) “Amino Acids, Peptides and Proteins in Organic Chemistry” (2009, Wiley-VCH, Weinheim, Germany), Jones “Amino acid and peptide synthesis” (2002, Oxford University Press) or “Engineering the genetic code” by Budisa (2005, Wiley-VCH, Weinheim, Germany) and obtained from standard suppliers of amino acids as mentioned above.

The peptides of the invention may be obtained from Open Biosystems (Huntsville, Ala., USA), Phoenix Pharmaceuticals (Burlingame, Calif., USA) or expressed and identified as described in US 2010/0056455, the disclosure of which is incorporated herein by reference. The peptides may also be synthesised by standard peptide synthesis techniques known to a person skilled in the art and described elsewhere, for example in “Chemistry of peptide synthesis” by Benoiton (2006, Taylor & Francis/CRC Press, London, UK), “Peptide synthesis protocols” by Pennington (1994, Humana Press, New York, N.Y., USA) and “Peptide synthesis and applications” by Howl (2005, Humana Press, New York, N.Y., USA).

A “subject” or “patient” of the invention may be a human or animal suffering or not suffering from any anxiety or sleep disorder or any other disease or symptom mentioned herein.

“Nasal/intranasal administration/application” in the sense of the invention denotes the delivery of a peptide or a pharmaceutical composition of the invention to the nose, nasal mucosa or the nostril of a subject in such a way that the peptide is able to arrive at the nasal mucosa or is able to contact with the nasal mucosa to pass the mucosal barrier/cells and finally be delivered to or arrive at the neuropeptide S receptor so as to exhibit the desired activity and to bring about the desired effect e.g. in causing, promoting, increasing, in the treatment of an anxiety or sleep disorder or as agonist of a receptor mentioned herein. The nasal administration of a pharmaceutical formulation or peptide of the invention is very convenient and easy to apply for the subject to be treated. Further, it is expected that nasal administration of the NPSR agonist leads to fewer immunological problems for the subject than other modes of administration. Moreover, intranasal or nasal administration of the peptide of the invention, such as NPS, differs e.g. from transmucosal administration in that it may comprise fast nose to brain delivery of the peptide (e.g. within 30 min or one hour) due to a combination of transmucosal and transneural administration, e.g. via the olfactory nerve. However, transmucosal administration alone without transneural administration via the olfactory nerve is not a nasal or intranasal administration within the meaning of the present application. A “fast nose to brain delivery” of the peptide within the meaning of the present invention may be a delivery from the nose to the brain or the neuropeptide S receptor within 120 min or less, 90 min or less, 60 min or less, 30 min or less or even 15 min or less.

The pharmaceutical compositions for nasal administration comprise at least one of the aforementioned peptides for use of the invention. For example, a pharmaceutical composition may also comprise at least two or at least three or more of the aforementioned peptides for use of the invention.

The pharmaceutical compositions described herein can be used for nasal administration, i.e. as a nasal medicament, to cause, promote or increase arousal, awakening, alertness, activity, spontaneous movement, anxiolytic effects or a combination thereof in a subject as well as to relieve or heal avoidance anxiety, dissociative anxiety such as flashbacks, depersonalisation, derealisation and intrusions, vegetative symptoms related to anxiety symptoms, especially in panic attacks, or a combination thereof in a subject. All these aforementioned modes of behaviour are to be understood according to their general meaning and in particular according to their meaning in behavioural studies.

Subjects or patients, which are in the need of a pharmaceutical composition to cause, promote or increase arousal, awakening, alertness, activity, spontaneous movement, an anxiolytic effect or a combination thereof as well as to relieve or heal avoidance anxiety, dissociative anxiety such as flashbacks, depersonalisation, derealisation and intrusions, vegetative symptoms related to anxiety symptoms, especially in panic attacks, or a combination thereof, are usually also in need of suitable medication such as the pharmaceutical composition of the invention in the prophylaxis and/or treatment of an anxiety or sleep disorder. Optionally said anxiety disorder is a disorder selected from the group consisting of panic disorder with and without agoraphobia, phobia, such as animal phobia, social phobia, height anxiety, claustrophobia and agoraphobia, posttraumatic stress disorder, generalised anxiety disorder, any other disease correlated with symptoms of pathological anxiety, and combinations thereof. Optionally said sleep disorder is a disorder selected from the group consisting of insomnia, hypersomnia, narcolepsy, idiopathic hypersomnia, excessive amounts of sleepiness, lack of alertness, lack of attentiveness, absentmindedness and/or lack of or aversion to movement or exercise, and combinations thereof.

Both the peptides and pharmaceutical formulations of the invention may be used to treat acute conditions and also chronic conditions. “Treatment” or “to treat” a patient in the sense of the invention are to be understood according to its meaning in the art, in particular according to its meaning in medicine and pharmacy. In general, a patient already suffering from an anxiety or sleep disorder or any symptom mentioned herein is treated in the sense of the invention in that anxiolysis (including reduction of avoidance and dissociative anxiety), arousal, awakening, alertness, activity, spontaneous movement, an anxiolytic effect or a combination thereof in the patient is caused, promoted or increased, thereby reducing or diminishing the symptoms mentioned herein and/or also healing, alleviating or curing an anxiety or sleep disorder of the patient.

“Prophylaxis” denotes that an anxiety or sleep disorder or any symptom mentioned herein is prevented to occur in a patient. To “prevent” in the sense of the invention denotes that an anxiety or sleep disorder or any symptom mentioned herein does not occur or is diminished or reduced or decreased in a patient. Thus, prophylaxis or a prophylactic treatment may be performed at a patient already suffering from an anxiety or sleep disorder or any symptom mentioned herein to prevent a new disorder or symptom to occur or to prevent an anxiety or sleep disorder or any symptom mentioned herein to occur in a patient which is regarded as healthy with respect to an anxiety or sleep disorder or any symptom mentioned herein. One example for a patient which is regarded as healthy and could be in the need of a prophylactic treatment may be a patient having a certain genetic disposition or only slight symptoms of fear, weakness or tiredness which would not be regarded as disorder or symptom in the medical sense.

The pharmaceutical compositions of the invention may be in any form suitable for nasal administration of one or more peptides to the nose of a human or animal. E.g., the pharmaceutical composition of the invention is in the form of a nasal spray, nose drops, nose ointment, nose powder or nose oil. In liquid compositions, a typical liquid carrier is water with the peptide being dispersed or dissolved in the water or Ringer solution. The pharmaceutical composition of the present invention may exist in various forms, for example, an oil-in-water emulsion, a water-in-oil emulsion and a water-in-oil-in-water emulsion. The pharmaceutical compositions may further comprise a pharmaceutically acceptable compound, an enhancer, a bacterial component, a biological compound, a protein, another peptide or a combination thereof. None of the other components in the pharmaceutical compositions, such as the pharmaceutically acceptable compound, the adjuvant, bacterial component, biological compound, the protein, other peptide or combinations thereof should diminish or decrease the activity of the peptides of the invention to bind to its receptor or lead to a degradation or truncation of the peptide as long as not wanted by the manufacturer. The latter may optionally be the case when a peptide of the invention in its active form is not very stable to different influences such as temperature, chemicals, light, etc. and thus may be present in the pharmaceutical composition in a “protected form”, i.e. comprise additional amino acids at its N- or C-terminus (i.e. an N- or C-terminal blocking group), additional glycostructures or other compounds which add to the peptide via hydrophobic interactions or van-der-Waals interactions. To remove these “protection compounds”, chemical or biological (e.g. proteases or glycosidases) compound may be present which selectively and/or in a slow mode remove the protection compounds.

A “pharmaceutically acceptable compound” denotes any liquid, solid or gaseous chemical or biological compound which is acceptable in a pharmaceutical composition or formulation characterised by good tolerability by a subject, being usually pharmacologically inactive or having no harmful effect on the physiology of the recipient. At least one, two, three, four, five or even more different pharmaceutically acceptable compounds may be present in a pharmaceutical composition of the invention, each in different amounts. The amount may be adjusted by the manufacturer according to the specific needs of the subject who is in need of a pharmaceutical composition of the invention or according to a dosage regimen. Examples of pharmaceutically acceptable compounds include drug entities, emulsifying agents, carbohydrates, lipids, panthenol, vitamins, caffeine, minerals, hyaluronic acid, trace elements, nucleic acids, calcium phosphate, water and oils, sodium chloride and other inorganic salts, magnesium, zinc, chamomile extract, buffering agents, such as phosphate buffer, phosphate buffered saline, succinate buffer or acetate buffer, such as sodium acetate, to result in a pH wherein the particular peptide is delivered optimally, such as a physiological pH or a pH in the range from 6.0 to 8.0, e.g. in the range of 6.5 to 8, or in the range of 7.0 to 7.5, or at pH 7.4±0.1, co-carriers, such as glycerol, glycine, propylene glycol, polyethylene glycols of various sizes, amino acids, a nasal mucosa permeation enhancer (e.g. a substance that enhances the permeation of the pharmaceutically active peptide composition through the nasal mucosa like quinidine or hyaluronic acid or inhibitors of the nasal mucosa peptidases) and other suitable soluble excipients, as is known to those who are proficient in the art of compounding of pharmaceutics.

An “enhancer” is used to improve the delivery of the peptide to a targeted area, i.e. enhances the transfer through the mucosa such as those described in U.S. Pat. No. 5,023,252. The pH of the pharmaceutical composition of the invention is typically in the range of physiological pH or a pH in the range from 6.0 to 8.0, or in the range of 6.5 to 8, or in the range of 7.0 to 7.5, or at pH 7.4±0.1.

Examples for an emulsifying agent are acacia, tragacanth, agar, pectin, carrageenan, gelatine, lanolin, cholesterol, lecithin, methylcellulose, carboxymethylcellulose, acrylic emulsifying agents, such as carbomers and combinations thereof. The emulsifying agent may be present in the pharmaceutical composition in a concentration that is effective to form the desired liquid emulsion. In general, the emulsifying agent may be used in an amount of about 0.001 to about 5 weight % of the pharmaceutical composition, or in an amount of about 0.01 to about 5 weight % of the pharmaceutical composition, or in an amount of about 0.1 to about 2 weight % of the pharmaceutical composition.

A “biological compound” may be any biological compound such as a carbohydrate, amino acid, lipid, nucleic acid, protein, peptide, cell compartment, phospholipids, polyether, plant, animal, or microbial compound.

“Lipids” are at least partially water-insoluble biological compounds due to a long hydrophobic carbohydrate part. Lipids are very important party of cell membranes in biological systems.

Examples of minerals comprised in the probiotic formulation of the invention are magnesium, calcium, zinc, selenium, iron, copper, manganese, chromium, molybdenum, potassium, vanadium, boron, titanium. In one embodiment, magnesium and/or calcium are present.

A “trace element” is a chemical element which is only needed in very low quantities for the growth, development and/or physiology of the organism, preferably of a human organism.

“Carbohydrates” are organic compounds consisting only of carbon, hydrogen and oxygen and having the empirical formula Cₘ(H₂O)ₙ, wherein the hydrogen to oxygen atom ratio is 2:1.

Examples of vitamins which may be comprised in the pharmaceutical composition of the invention are water-soluble and water-insoluble vitamins, such as vitamin A (e.g. retinol, retinal and carotenoids including beta carotene), vitamin B₁ (thiamine), vitamin B₂ (riboflavin), vitamin B₃ (e.g. niacin, niacinamide, nicotinamide), vitamin B₅ (pantothenic acid), vitamin B₆ (e.g. pyridoxine, pyridoxamine, pyridoxal) vitamin B₇ (biotin), vitamin B₉ (e.g. folic acid, folinic acid), vitamin B₁₂ (e.g. cyanocobalamin, hydroxycobalamin, methylcobalamin), vitamin C (ascorbic acid), vitamin D (e.g. ergocalciferol, cholecalciferol), vitamin E (e.g. tocopherols, tocotrienols), vitamin K (e.g. phylloquinone, menaquinones) and mixtures thereof.

A “bacterial component” denotes a compound, such as a biological molecule, a polysaccharide, lipid or else of bacterial origin or being produced by bacterial fermentation or expression.

Another peptide which may be present in the pharmaceutical formulation may be a neuropeptide, anti-inflammatory peptide, endorphin, growth hormone, growth hormone releasing hormone, leptin or a fragment or a combination thereof.

The peptide of the invention may be present in the pharmaceutical composition in a therapeutically suitable concentration. A “therapeutically suitable concentration” in the sense of the invention is a concentration which allows the nasal administration of the peptide in a therapeutically effective amount in a general application size or volume. A “therapeutically effective amount” is an amount which results in or leads to the desired effect in a patient, e.g. that relieve or healing of avoidance anxiety, dissociative anxiety such as flashbacks, depersonalisation, derealisation and intrusions, vegetative symptoms related to anxiety symptoms, especially in panic attacks, or arousal, awakening, alertness, activity, spontaneous movement, an anxiolytic effect or a combination thereof in the patient is caused, promoted or increased, and/or the symptoms mentioned herein are reduced or diminished or the anxiety or sleep disorder is healed, alleviated or cured. The skilled person knows, however, that the desired effect may occur immediately or only after treatment over a period of days, weeks or months. It may also be the case that the desired effect has to be maintained by regular nasal administration of the peptide or pharmaceutical formulation of the invention. The skilled person also knows that the concentration may be less than the most optimal therapeutically effective amount (which would correspond to a concentration which results in best treatment or prophylaxis results) due to possible side effects in the patient and/or allergic reactions of the patient. The therapeutically effective amount and therefore also the therapeutically suitable concentration depends on the form in which the pharmaceutical composition is administered. In case of a nasal spray, the concentration may be adjusted to the volume of a single or two spray events per application. The same may account for the volume and number of nose drops, as well as an amount of nose ointment, nose powder or nose oil which may typically be administered in a single application. Beside the concentration of the peptide in the pharmaceutical composition the number and repeats of administration may also be increased or reduced depending on the fitness of the patient and the severity of the disorder or symptoms. An example of a typical therapeutically effective amount of the peptide which may be administered to the subject in a single application is in the range of 0.05 μg to 200 μg, 0.1 μg to 100 μg, 0.5 μg to 75 μg, 1 μg to 50 μg, 2 μg to 40 μg, 3 μg to 30 μg, 4 μg to 25 μg, 5 μg to 20 μg, 5 μg to 15 μg, or in the range of 5 μg to 10 μg. Further examples of a typical therapeutically effective amount of the peptide which may be administered to the subject in a single application is 0.05 μg, 0.1 μg, 0.5 μg, 0.75 μg, 1 μg, 1.5 μg, 2 μg, 2.5 μg, 3 μg, 3.5 μg, 4 μg, 4.5 μg, 5 μg, 6 g, 7 μg, 7.5 μg, 8 μg, 9 μg, 10 μg, 12.5 μg, 15 μg, 17.5 μg, 20 μg, 25 μg, 30 μg, 40 μg, 50 μg, 75 μg, 100 μg or 150 μg. An example of a typical therapeutically suitable concentration of the peptide in the pharmaceutical composition is about 0.0001 to about 10 weight % of the pharmaceutical composition, optionally an amount of about 0.0005 to about 5 weight % of the pharmaceutical composition or an amount of about 0.001 to about 2 weight % of the pharmaceutical composition. Another exemplary concentration of the peptide in the pharmaceutical composition is in the range from 0.01 μg/mL to 50 mg/mL, optionally from 0.05 μg/mL to 20 mg/mL, or from 0.1 μg/mL to 10 mg/mL, or 0.5 μg/mL to 5 mg/mL, or 0.75 μg/mL to 1 mg/mL, or from 1 μg/mL to 500 μg/mL, from 2.5 μg/mL to 250 μg/mL, from 5 μg/mL to 150 μg/mL, or from 10 μg/mL to 125 μg/mL, or from 15 μg/mL to 100 μg/mL, or from 20 μg/mL to 100 μg/mL.

The pharmaceutical formulation may be administered every 30 min, every hour, every second hour, every third hour, once, twice, three times, four times, five or six or seven or eight times per day, every second, third, fourth, fifth, sixth day, weekly, monthly, every three months or every six months or yearly. The number and time of administration may be adjusted according to the physician's recommendation and according to the patient's fitness and severity of the disorder or symptoms. The administration may also be different each day, week, month or year depending on the specific requirements of the patient. It may for example be necessary to start the nasal treatment with a high dose and in short intervals which may be reduced to a lower dose or frequency after reduction of most symptoms or after reduction of the severity of the disorder or symptom.

Further provided is the use of the peptide or the pharmaceutical composition of any embodiment described herein for nasal application to a subject.

The invention also provides a method for identifying intracerebral target neurons of an intranasally applied peptide in an animal, wherein the peptide is administered nasally. Said peptide may optionally comprise a fluorescence tag or a fluorescence amino acid. In one embodiment, the method is for identifying the target neurons of any peptide of the present invention mentioned herein or of a peptide which is comprised in any pharmaceutical composition of the invention. Said method may comprise one or more of the following steps: a step of administering the peptide or the pharmaceutical composition nasally to the animal, optionally in a therapeutically effective amount, a step of sacrificing of the animal, and a step of animal brain removal and perfusion for histological examination.

By conducting the method for identifying intracerebral target neurons as described herein it can be shown that the peptides and pharmaceutical composition of the invention are suitable for nasal administration to a subject, patient, human or animal. In addition or alternatively, other peptides may be identified which have similar activity based on a similar or comparable brain activation pattern.

The animal may be selected from the group consisting of a mammal, e.g. a rodent (e.g. mouse, guinea pig, rat, rabbit), cat, dog, pig, chimpanzee, a bird (e.g. chicken, duck, goose), horse, pony, cattle and others. The immunohistochemical preparation and examination may be performed as follows: removal of the brains, post-fixing of the brains in 4% formaldehyde, brain cryoprotection in 20% sucrose, shock-freezing of the brains in methylbutane. Immunohistochemistry may then be performed on free-floating cryosections (e.g. having 40 μm). Suitable fluorescence-tagged antibodies for immunostaining are for example: (i) primary antibodies, e.g. for neurofilament (1:1000; Abcam, Cambridge, UK) and GFAP (1:250; DAKO, Glostrup, Denmark), and (ii) secondary antibodies, depending on the first antibody e.g. mouse-, rabbit- or rat-specific, (Alexa 488 goat anti-mouse IgG) or rabbit (Alexa 488 donkey anti-rabbit IgG) (1:300; Invitrogen, Leek, The Netherlands). The sections may then be counterstained with DAPI (200 ng/ml; Carl Roth, Karlsruhe, Germany) and mounted with a fluorescence-preserving mounting medium (Shandon Immu-Mount, Thermo Scientific, Waltham, Mass., USA).

The invention is further described by the following examples which are solely for the purpose of illustrating specific embodiments of the invention, and are not to be construed as limiting the scope of the invention in any way.

### Examples

### 1. Animals

For behavioural experiments, C57BL/6N males were purchased from Charles River Germany GmbH (Sulzfeld, Germany). Male HAB mice were obtained from the animal facility of the Max Planck Institute (MPI) of Psychiatry (Munich, Germany). For all other animal experiments, C57BL/6N males bred in the animal facility of the MPI of Biochemistry (Martinsried, Germany) were used. Experiments were performed with 10 week-old animals. All procedures were approved by the Government of Upper Bavaria and were in accordance with European Union Directive 86/609/EEC.

### 2. Administration of Fluorophore-Labelled NPS

For ICV injection, a guide cannula was implanted into the right ventricle using a stereotaxic frame (coordinates: 0.3 mm caudal and 1.1 mm lateral from the bregma; 1.3 mm ventral from the skull surface). 8 days later, mice were injected with 2 μL of Cy3-NPS) or rhodamine-NPS (both 10 μM, both Phoenix Pharmaceuticals, Burlingame, Calif., USA) or pure rhodamine (1 g/ml, Sigma-Aldrich, St. Louis, Mo., USA). Mice were sacrificed at 2, 10 or 30 min post-injection. To clarify the internalisation mechanism of NPS, 2 μL of native NPS (50 μM or 100 μM, rat, Bachem, Bubendorf, Switzerland) in Ringer solution were pre-injected 10 min before injection of Cy3-NPS. The mice were sacrificed 30 min post-injection. For intranasal application, the anesthetised mice were placed in a supine position, with the head supported at a 450 angle to the body as reported elsewhere (van den Berg et al., 2002). 7 μL of Cy3-NPS (10 μM) or pure rhodamine (10 g/mL) were applied alternatingly to each nostril; after 5 min, the procedure was repeated. The mice were sacrificed at 15 min, 30 min and 4 hrs after the first application.

### 3. Immunohistochemistry

Brains were removed, post-fixed in 4% formaldehyde and cryoprotected in 20% sucrose, then shock-frozen in methylbutane. Immunohistochemistry was performed on free-floating cryosections (40 μm). The primary antibodies used were specific for neurofilament (1:1000; Abcam, Cambridge, UK) and GFAP (1:250; DAKO, Glostrup, Denmark). The secondary antibodies used were specific for mouse (Alexa 488 goat anti-mouse IgG) and rabbit (Alexa 488 donkey anti-rabbit IgG) (1:300; Invitrogen, Leek, The Netherlands). The sections were counterstained with DAPI (200 ng/ml; Carl Roth, Karlsruhe, Germany) and mounted with a fluorescence-preserving mounting medium (Shandon Immu-Mount, Thermo Scientific, Waltham, Mass., USA).

### 4. Behavioural Experiments

Behavioural tests (open field, dark-light test and elevated plus maze (EPM)) following intranasal NPS application were performed sequentially. Each test lasted for 5 min with an inter-test interval of 5 min, as described previously (Bunck et al., 2009; Krömer et al., 2005). The animals' behaviour during testing was videotaped and relevant parameters were analysed with the tracking software ANY-maze version 4.30 (Stoelting, Wood Dale, Ill., USA). Native NPS (1 μg/μL) or vehicle (Ringer solution) were administered intranasally to the alert mouse as described above (total volume: 14 μL). Mice were tested 30 min and 4 hrs after intranasal administration of first drop. Animals were sacrificed 24 hrs after treatment and prefrontal cortex and bilateral hippocampi were prepared immediately and shock-frozen in methylbutane.

### 5. Immunoblotting

For immunoblotting, proteins were extracted from aforementioned two brain regions (prefrontal cortex and bilateral hippocampi). Quantitative blot analysis was performed using ImageJ software (http://rsbweb.nih.gov/ij/; Rasband, W. S., ImageJ, U.S. National Institutes of Health). Primary antibodies used: Glt-1, Glu-R1, Glu-R2 (all 1:100; all from Santa Cruz Biotechnology, Santa Cruz, Calif., USA), synapsin (1:2000; Synaptic Systems, Goettingen, Germany) and GAPDH (1:2000, Santa Cruz Biotechnology, Santa Cruz, Calif., USA). Secondary antibodies used: donkey anti-goat IgG-HRP (1:10000; Santa Cruz Biotechnology, Santa Cruz, Calif., USA), goat anti-rabbit IgG-HRP (1:7500, Sigma-Aldrich, St. Louis, Mo., USA) and goat anti-mouse IgG-HRP (1:25000, Sigma-Aldrich, St. Louis, Mo., USA).

### 6. Image Acquisition and Processing

Images were acquired either with a confocal microscope (Olympus IX81, software: FluoView FV1000 2.1.2.5) or, in case of HEK-cells, with a fluorescence microscope (Olympus BX61, software: cell̂F 2.8, Olympus Soft Imaging Systems GmbH). After acquisition, the images were processed using Photoshop and Illustrator (Adobe, San Jose, Calif., USA).

### 7. Statistical Analysis

Statistical analysis was performed using GraphPad Prism 5.03 (GraphPad Software, Inc.). For analysis of the behavioural and immunoblotting data, the one-tailed and the two-tailed unpaired t-test were used, respectively. P-values below 0.05 were considered significant. For the behavioural data, Grubbs' test was used to identify and exclude outliers. For the electrophysiology data, the two-tailed unpaired Student's t-test was used.

### 8. Target Brain Regions and Target Cells of NPS

To identify NPS target cells in the murine brain, the distribution pattern of a fluorescent NPS-conjugate (Cy3-NPS) after unilateral ICV injection in Bl6 mice was examined. 10 minutes after ICV injection, single cells already exhibited distinct patterns of fluorescence (data not shown). 30 minutes after ICV-injection, various cell populations in distinct brain regions had internalised Cy3-NPS (cf. Table 2).

TABLE 2
Overview of brain regions targeted by Cy3-NPS.

Forebrain
Accumbens nucleus
Anterior olfactory area, dorsal part
Anterior olfactory area, external part
Basal ganglia
Globus pallidus
Cerebral cortex
Primary motor cortex
Secondary motor cortex
Somatosensory cortex
Cingulate cortex, area 1
Endopiriform cortex
Amygdala
Medial amygdaloid nuclei
Anterior cortical amygdaloid nuclei
Posterior cortical amygdaloid nuclei
Basolateral amygdala
Central amygdala
Lateral amygdala
Bed nucleus of the stria terminalis
(intraamygdaloid division)
Amygdalohippocampal area
Hippocampus
Dentate gyrus
CA1 CA2, CA3
Ventral hippocampus, granular layer
of dentate gyms
Thalamus
Medial habenula
Lateral habenula
Paraventricular thalamic nucleus
Mediodorsal thalamic nucleus
Hypothalamus
Arcuate nucleus
Paraventricular nucleus
Dorsomedial nucleus
Ventromedial nucleus
Periventricular nucleus
Suprachiasmatic nucleus
Preoptic area
Median preoptic nucleus
Ventromedial preoptic nucleus
Vascular organ of the lamina terminalis
Midbrain and brainstem areas
Dorsal raphe
Posterodorsal tegmental nucleus
Periaqeductal gray
Central gray of the pons
Red nucleus
Locus coeruleus
Barrington's nucleus
Medial parabrachial nucleus
Medial vestibular nucleus
Cerebellum
Purkinje cells

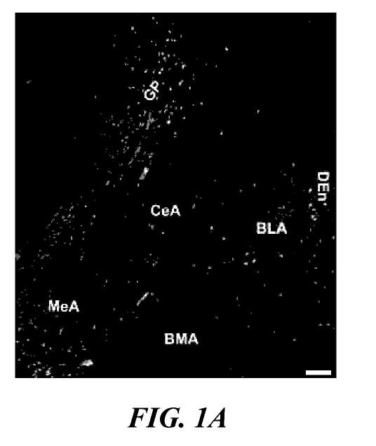

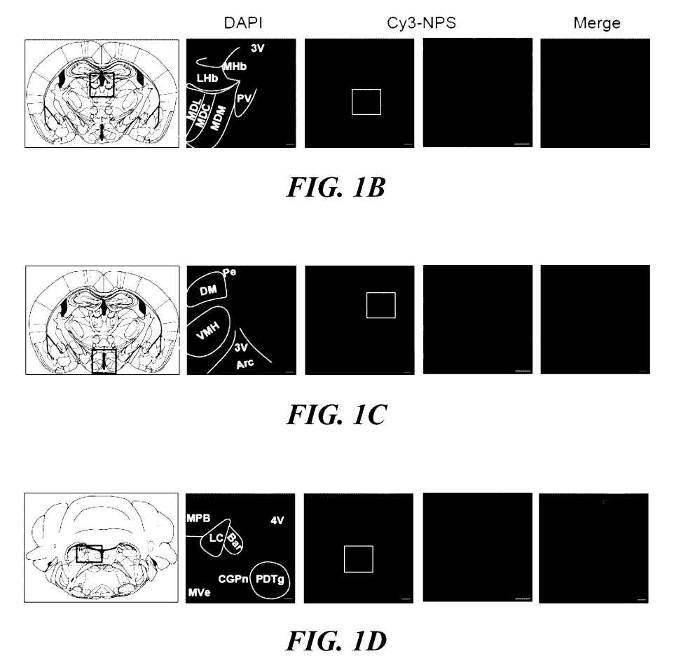

Cells containing Cy3-NPS were present within the basal ganglia (globus pallidus and nucleus accumbens) and also in amygdaloid nuclei, including the basolateral and central amygdala (FIG. 1D). Cy3-NPS was additionally found in other regions associated with stress-response and learning, such as the lateral habenula and the mediodorsal thalamic nuclei, respectively (FIG. 1A), as well as in regions with neuroendocrine functions, such as the arcuate and ventromedial hypothalamic nuclei (FIG. 1B). It also targeted single cells within the locus coeruleus, the tegmental nucleus, Barrington's nucleus and the parabrachial nucleus (FIG. 1C).

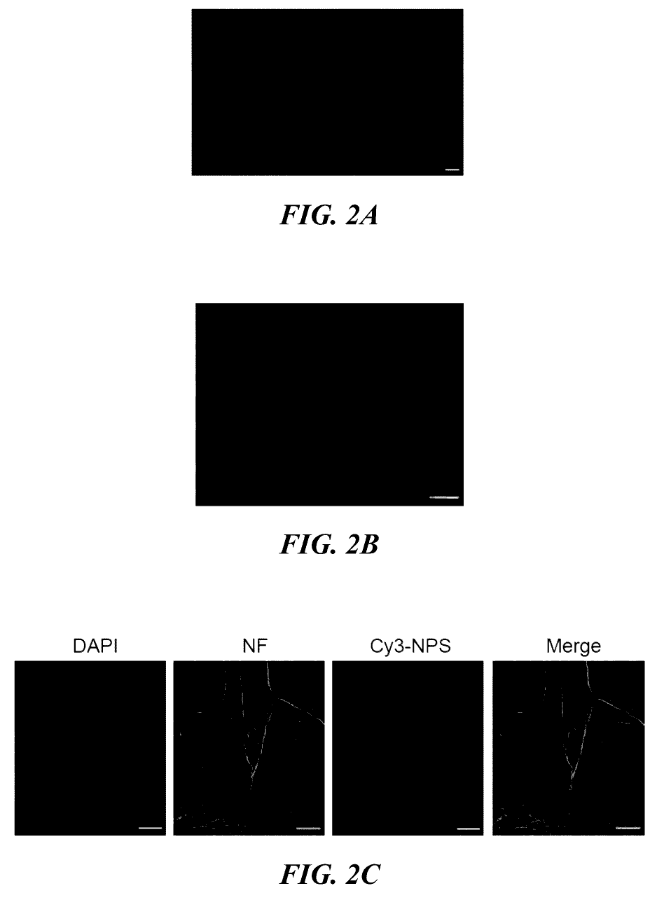

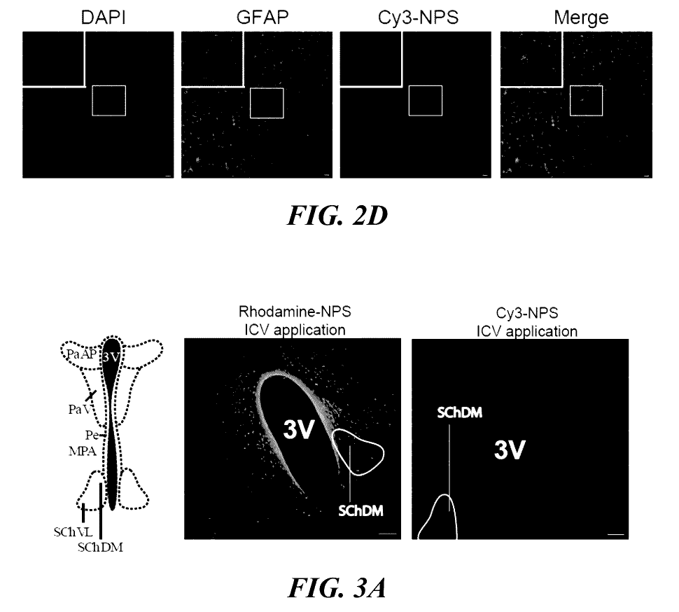

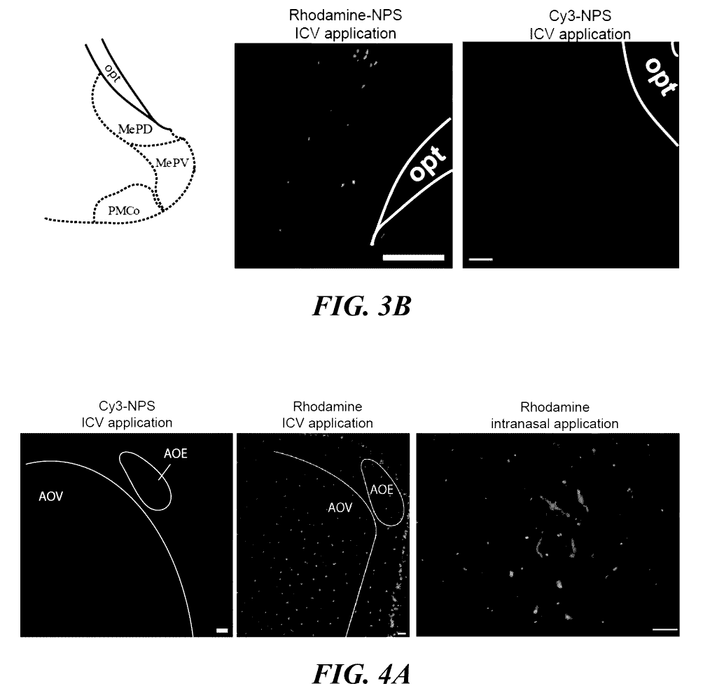

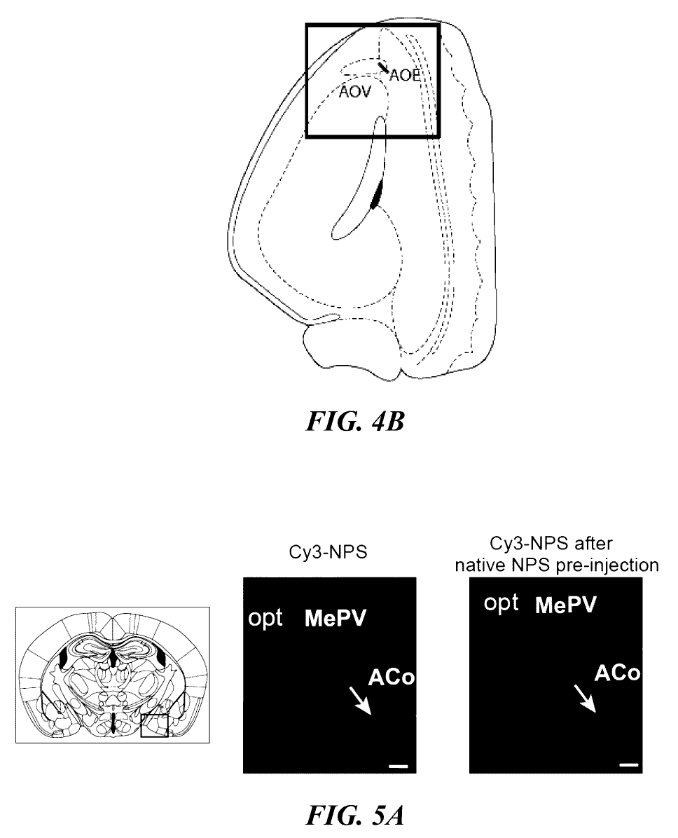

Most notable was the internalisation of Cy3-NPS in the hippocampal CA1, CA2 and CA3 regions, mainly in the pyramidal and oriens layers and sparingly in the radiate and molecular layers (FIG. 2A), as well as in the granulate and polymorph layers of the dentate gyrus (FIG. 2B). To test the specificity of the distribution pattern of ICV-injected NPS, it was compared to the distribution pattern of rhodamine-NPS and unconjugated rhodamine, respectively. Cy3-NPS and rhodamine-NPS were internalized specifically into certain cells and exhibited almost identical intracerebral distribution patterns, whereas pure rhodamine dispersed homogenously in the intercellular space throughout the entire brain, forming aggregates not corresponding to any cellular structures (FIGS. 3A, 3B, 4A and 4B). These findings indicate that the intracerebral distribution pattern described here is specific for native NPS but not for NPS-fluorophore fusion molecules nor for the unconjugated fluorophore. Cy3-NPS was found in the cytosol and throughout the processes of target cells (FIG. 2B). To characterise these cells, immunostainings against the neuronal marker neurofilament (NF) and the astroglial marker glial fibrillary acidic protein (GFAP) on brain sections from animals treated with Cy3-NPS were performed. Cy3-NPS co-localised exclusively with the neuronal marker (FIG. 2C). Additionally, cells containing Cy3-NPS possessed typical morphological features of neurons, being larger and exhibiting fewer processes than astroglia (FIG. 2D). Cells not expressing the neuronal marker did not take up Cy3-NPS. Taken together, it can be concluded that NPS is internalised exclusively into neurons.

### 9. Intracellular Uptake of Cy3-NPS is Mediated by Internalisation of the Receptor-Ligand Complex

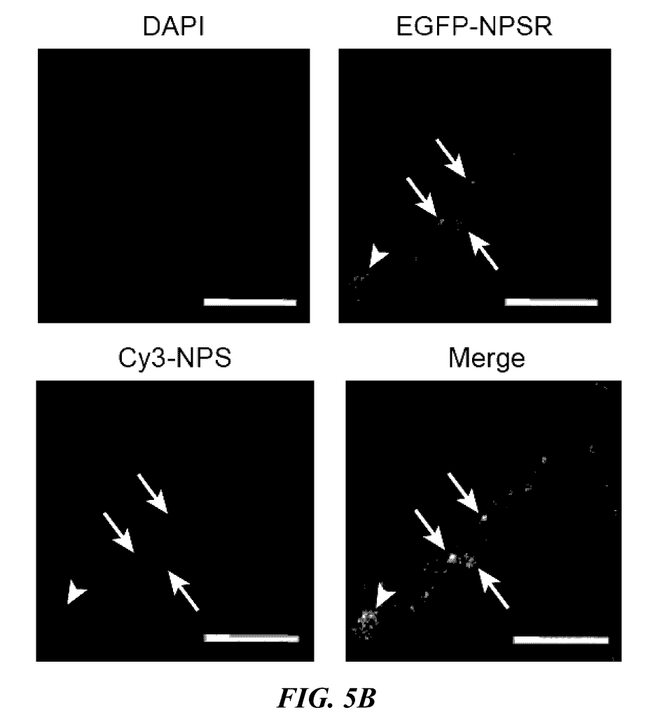

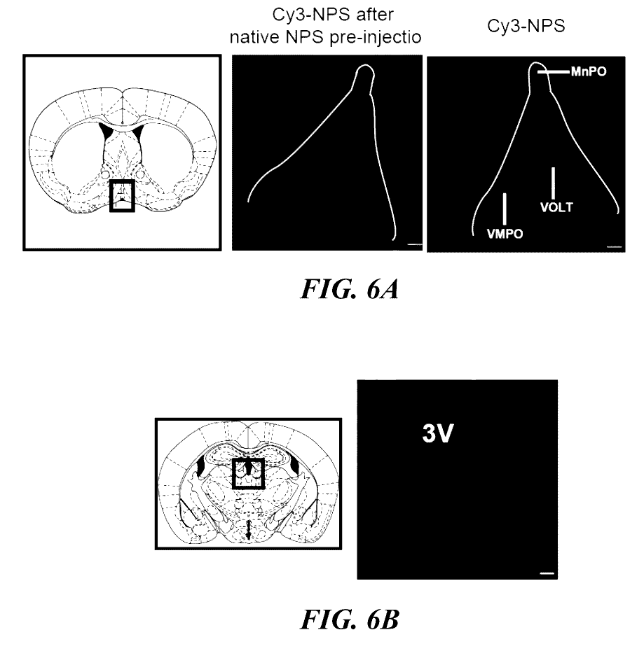

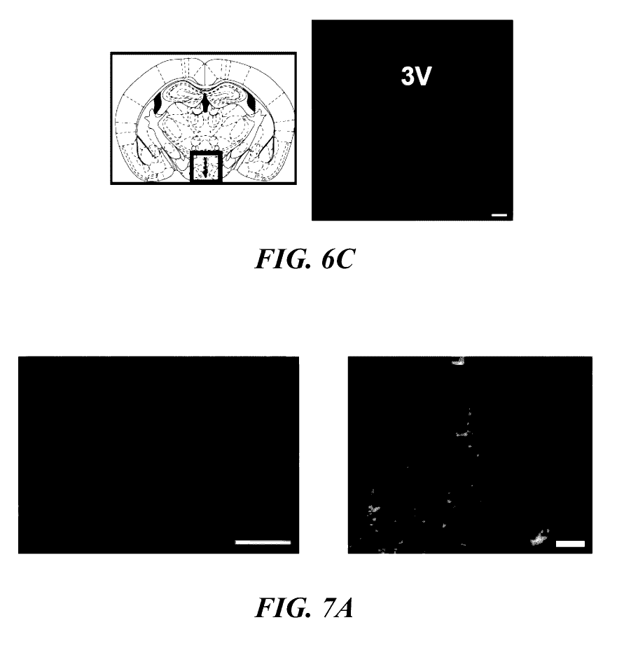

To clarify the mechanism of intracellular Cy3-NPS uptake, native, i.e. unlabeled, NPS at 5 fold concentration was injected unilaterally 10 min prior to ICV injection of Cy3-NPS (0.2 nmol per mouse). Pre-injection of native NPS reduced Cy3-NPS uptake throughout the brain (FIG. 5A and FIGS. 6A-6C). This points towards a receptor-mediated uptake mechanism, since, as shown for other neuropeptides (cf. Grady et al., 1996; Hubbard et al., 2009) pre-treatment with unlabeled agonists leads to receptor saturation, thereby antagonising the uptake of labelled agonist (here Cy3-NPS).

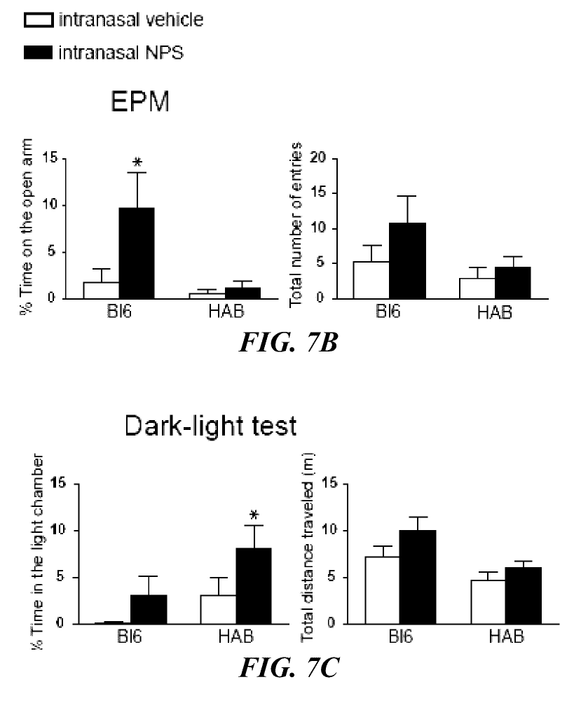

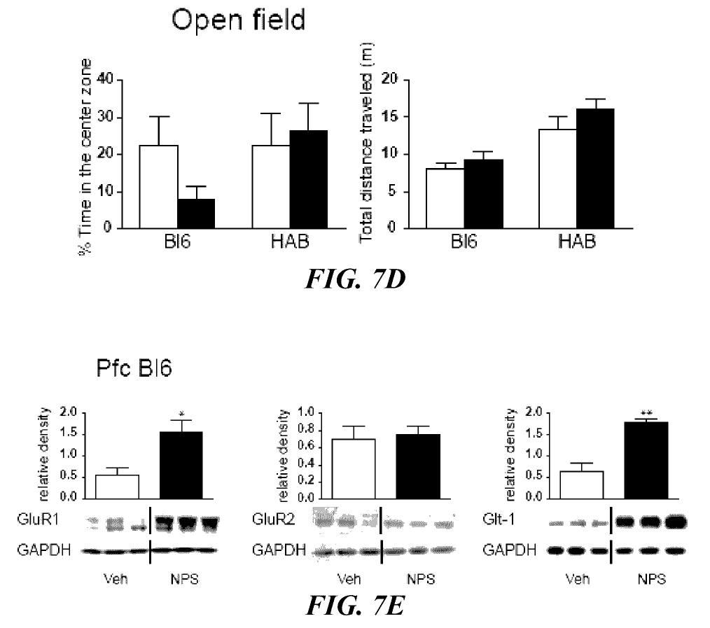

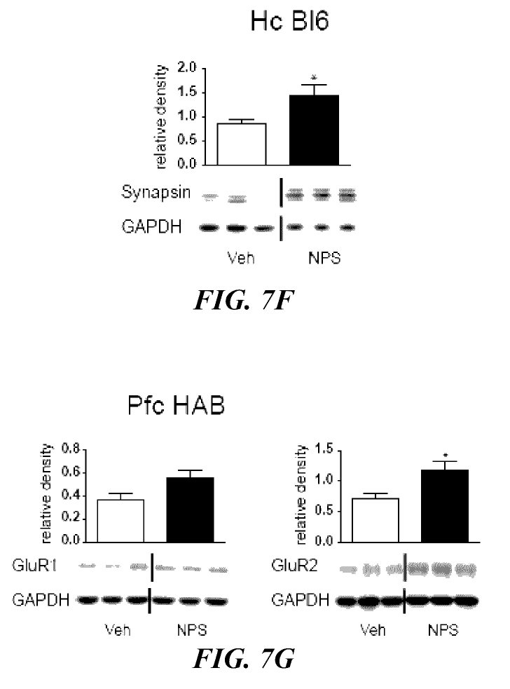

To show that Cy3-NPS internalisation is receptor-mediated, the cellular NPS uptake mechanism in cultured HEK cells incubated with Cy3-NPS after having been transiently transfected with EGFP-NPSR was studied. As co-localisation of Cy3-NPS and EGFP-NPSR indicates (FIG. 5B, panel “merge”), the receptor-ligand complex was internalised and subsequently accumulated in cytoplasmic and perinuclear vesicular structures. Surface expression of EGFP-NPSR is required for Cy3-NPS internalisation, since HEK cells transfected with an empty EGFP-expression plasmid did not take up Cy3-NPS molecules. In view of the fact that the NPSR is reported to be the only receptor mediating NPS effects (Duangdao et al., 2009) it may be concluded that the intracellular uptake of Cy3-NPS observed here also in vivo (FIGS. 1A-1D and FIG. 7A) likewise depends on the NPSR.

### 10. Intranasal Administration Delivers Cy3-NPS to its Target Cells

To establish a non-invasive NPS administration method also applicable in humans, the effectiveness of intranasal NPS administration in mice was investigated. First, a stress-free intranasal administration procedure for liquid substances in alert or anesthetised mice was designed (see Example item 2) and then cerebral distribution patterns of intranasally and ICV administered Cy3-NPS were compared. It was found that both patterns are identical at 30 min after NPS application. At this time point, Cy3-NPS distributes throughout the brain, from the olfactory bulb to caudal subcortical structures. There, it accumulates intraneuronally as after ICV injection (FIG. 7A). A distribution timeline of intranasally applied Cy3-NPS revealed that at 15 min post application intracellular Cy3-NPS uptake was visible only in the olfactory bulb, and that 4 hrs after application almost all traces of Cy3-NPS disappeared. Taken together, it was successfully demonstrated that intranasally administered NPS is effectively delivered to its brain target neurons in the living animal.

### 11. Nasally Administered NPS Exerts Strong Anxiolytic Effects on Bl6 and HAB Animals

After having ascertained that nasally administered NPS reaches its target cells, anxiolytic effects of NPS were established for this transnasal delivery method. For this purpose, nasally 6.36 nmol NPS per mouse was administered to two mouse strains, Bl6 and HAB (high anxiety behaviour) mice. HAB animals, a mouse strain inbred for pathologically high anxiety, were chosen together with Bl6 mice to allow differentiation between the temporary condition of state anxiety and the general condition of trait anxiety (Bunck et al., 2009; Kromer et al., 2005). To accurately characterise the anxiety-relieving properties of NPS in these two mouse strains, three standardised behavioural assays measuring anxiolytic effects at two different time points after intranasal NPS administration were performed. In each test, the effects of intranasal NPS versus vehicle treatment on both anxiety- and locomotion-related indices were examined.

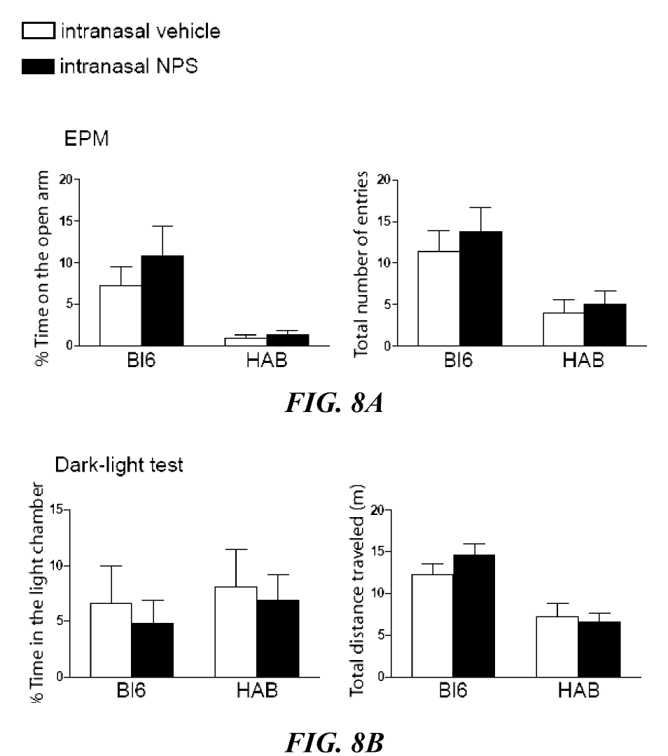

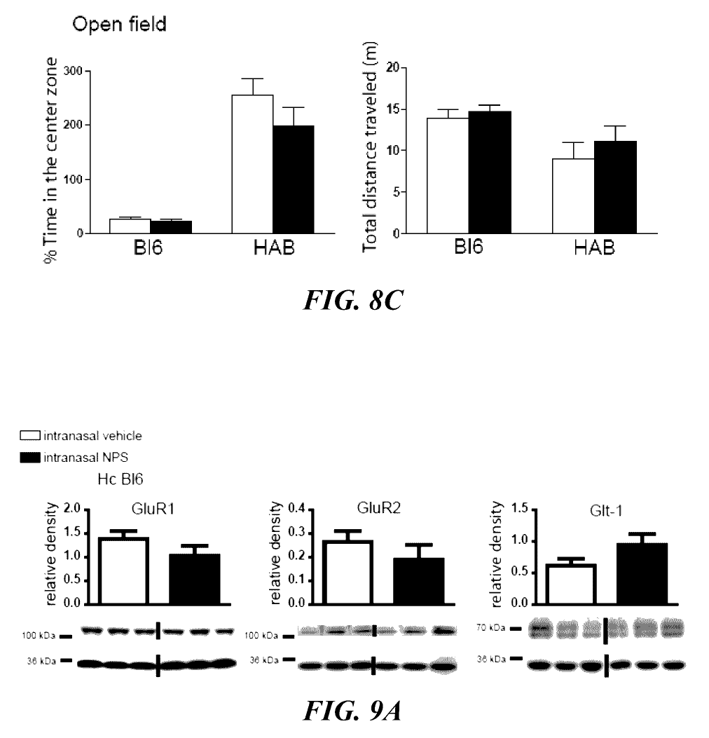

At 30 min after administration, there were no behavioural differences between NPS- and vehicle-treated animals in any of the three tests performed (FIGS. 8A-8C). This applied to both mouse strains tested, Bl6 as well as HAB mice. However, at the later time point of 4 hrs after intranasal administration, NPS-treatment elicited behavioural effects in both Bl6 and HAB mice. Among Bl6 animals challenged in the elevated plus maze (EPM), NPS-treated individuals significantly increased their time on the open arms, whereas there was no difference between treatment and control in the total number of entries (FIG. 7D). In the dark-light test, NPS-treated and control animals did not differ significantly in the time spent in the light chamber or in the total distance traveled (FIG. 7C). Similarly, in the open field, NPS treatment had no effect on any parameter examined (FIG. 7B). In HAB mice, NPS-treatment significantly increased the time spent in the light chamber during the dark-light test 4 hrs after administration, leaving the total distance traveled unaffected (FIG. 7C). There were no differences neither in the EPM nor in the open field in any parameter tested (FIG. 7B and FIG. 7D). In summary, 4 hrs after administration, nasal administered NPS led to increased time on the open arms in the EPM in Bl6 mice and to increased time in the light chamber in the dark-light test in rigidly predisposed HAB mice. No differences were detected in the total distances traveled in any of the tests, indicating locomotion-independent anxiolytic effects induced by intranasal NPS administration.

### 12. Nasally Administered NPS Upregulates Cerebral Proteins Involved in the Glutamatergic Network and in Synaptic Function

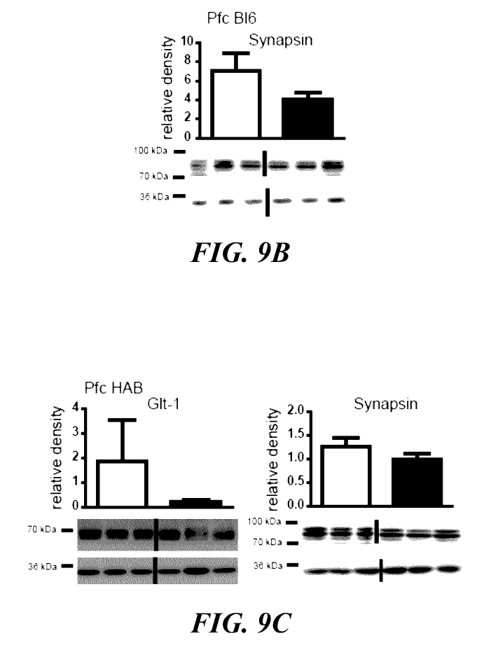

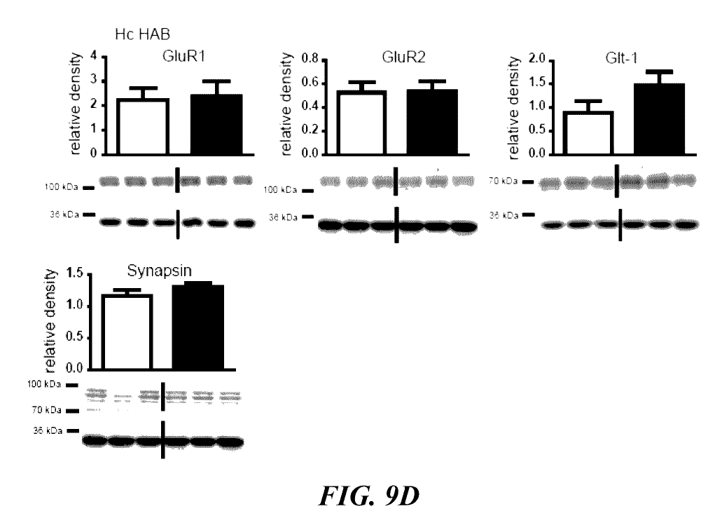

Although the behavioural effects of NPS have been well documented, its effects on cerebral protein expression have hitherto not been studied. Therefore, candidate proteins for immunoblot examination relying on publications linking NPS to the glutamatergic system (Han et al., 2009; Okamura et al., 2010) and to synaptic function (Jüngling et al., 2008; Raiteri et al., 2009) were selected. Expression levels of proteins involved in the glutamatergic network such as subunits of AMPA receptors and glutamate transporters, and synapsin, a protein involved in synaptic formation and function, were examined 24 hrs after intranasal NPS treatment by immunoblotting of lysates prepared from both the prefrontal cortex and the hippocampus. Among Bl6 animals, NPS treatment significantly increased expression levels of the subunit 1 of the AMPA receptor (GluR1) and of the glutamate transporter type 1 (Glt-1) in the prefrontal cortex, whereas expression of the subunit 2 of the AMPA receptor (GluR2) remained unchanged (FIG. 7E). Expression of these proteins remained unaffected in the hippocampus (FIG. 9A). On the other hand, synapsin expression significantly increased in the hippocampus only (FIG. 7G and FIG. 9C), indicating region-specific regulatory effects of NPS. In the prefrontal cortex of HAB mice, a significantly increased expression of GluR2 after NPS treatment was detected while expression of GluR1 was not affected (FIG. 7F). Glt-1 levels also remained unchanged (FIG. 9D). These findings were region-specific, with no corresponding changes in the hippocampus (FIG. 9B).

### 13. Direct Involvement of the Ventral Hippocampus in Neuropeptide S-Induced Anxiolysis

### 13.1 Animals

All experiments were performed in adult (10- to 12-week-old) male mice. For behavioural experiments, C57BL/6N mice were purchased from Charles River Germany GmbH (Sulzfeld, Germany). For all other experiments, C57BL/6N animals bred in the animal facility of the Max Planck Institute (MPI) of Biochemistry (Martinsried, Germany) were used. High-anxiety behaviour (HAB) mice were obtained from the animal facility of the MPI of Psychiatry (Munich, Germany). All animals were housed individually for at least 6 days before the start of experiments, on a 12 h light/dark cycle with food and water ad libitum. All procedures were approved by the Government of Upper Bavaria and were in accordance with European Union Directive 86/609/EEC.

### 13.2 Chemicals

Cy3-NPS was purchased from Phoenix Pharmaceuticals (Karlsruhe, Germany) and rat NPS from Bachem (Bubendorf, Switzerland). Both were dissolved at the desired final concentration in artificial cerebrospinal fluid (ACSF, for composition see below). Di-4-ANEPPS and all salts for the ACSF were purchased from Sigma Aldrich (Taufkirchen, Germany). A 20.8 mM stock solution of Di-4-ANEPPS was prepared in DMSO. The active enantiomer of the specific NPSR antagonist SHA 68, (R)-SHA 68 (Okamura et al., 2008; Trapella et al., 2011), was from A. Sailer (Novartis, Basel, Switzerland). (R)-SHA 68 was dissolved in DMSO and diluted for use in ACSF at a final concentration of 10 μM (<0.1% DMSO).

### 13.3 Surgery

Animals were fixed in a stereotactic frame and maintained under isoflurane anesthesia (Forene® 100%, V/V; induction: 2.5%; maintenance: 1.5%; in O₂; flow rate: 1 L/min). The mice received acute analgesic treatment with Metacam s.c. during surgery (0.5 mg/kg; in NaCl). 23 gauge stainless-steel guide cannulas were implanted in the CA1 region of the VH at the following coordinates: 3.1 mm posterior, ±3 mm lateral from bregma, and 2 mm ventral from the skull surface (Franklin and Paxinos, 2007). The guide cannulas were fixed with two screws and a two-component adhesive. For behavioural experiments, animals were implanted bilaterally for later bilateral injection, whereas for Cy3-NPS injections, implantation was performed unilaterally. The animals were allowed to recover for at least 6 days before starting the behavioural experiments. Substance infusions were carried out manually, on mice anesthetised by brief inhalation of isoflurane, using a 30 gauge injection cannula connected to a Tygon tube and a 10 μL Hamilton syringe. After infusion, the injection cannula was kept in place for additional 30 s to prevent substance outflow.

### 13.4 Administration of Cy3-NPS and Brain Section Processing

Cy3-NPS was administered unilaterally at a concentration of 0.07 nmol in a volume of 0.7 μL ACSF. The mice were sacrificed 30 min after application. Brains were removed and post-fixed in 4% paraformaldehyde overnight at 4° C., then shock-frozen in methylbutane and stored at −80° C. 40 am cryosections were cut from the olfactory bulb until the first third of the cerebellum. Then, the sections were thaw-mounted and counterstained with 4′,6-diamidin-2-phenylindole (DAPI, 200 ng/mL, Carl Roth, Karlsruhe, Germany). After mounting with a fluorescence-preserving medium (Shandon Immu-Mount, Thermo Scientific, Bonn, Germany), sections were stored at 4° C. Images were acquired with a confocal microscope (Olympus IX81, software: FluoView FV1000 2.1.2.5).

### 13.5 Behavioural Experiments

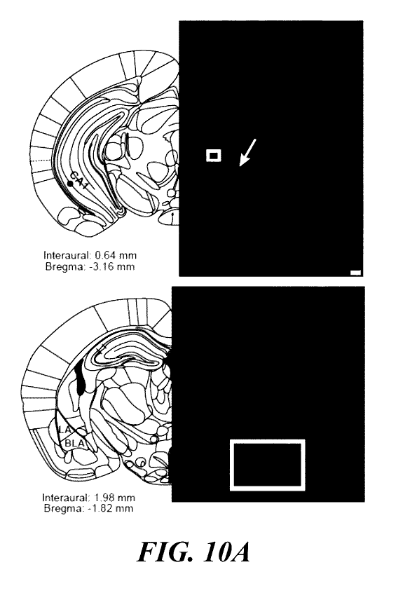

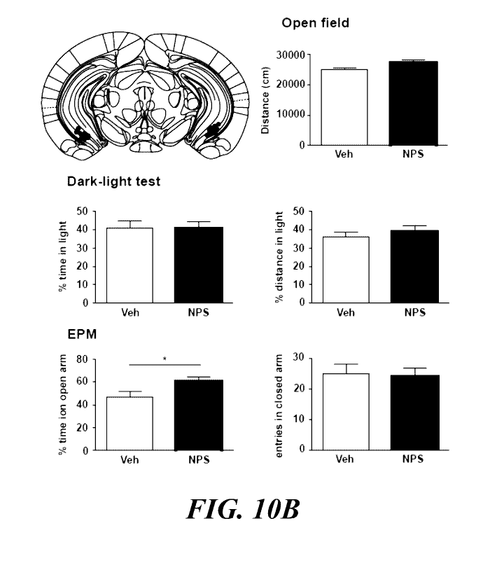

Mice were injected bilaterally either with 0.1 nmol NPS in 0.5 μL ACSF for each side or with 0.5 μL of ACSF for each side. 30 min after injection, three behavioural assays [open field, dark-light test, and elevated plus maze (EPM)] were performed sequentially in the order mentioned. Each test lasted 5 min, with a 5 min break in between, as described in Kromer et al., 2005 and Bunck et al., 2009. Animal behaviour was videotaped and relevant parameters were analysed using the tracking software ANY-maze version 4.30 (Stoelting, Wood Dale, Ill., USA). Mice were sacrificed 24 h after completion of behavioural assays and the locations of the guide cannulas were checked in histological cryosections of 40 μm counterstained with DAPI. The implantation and injection sites are shown in FIG. 10B. Mice with deviating injection sites were excluded from further analysis.

### 13.6 Intranasal Administration of NPS

Intranasal administration of NPS was performed as described above in item 2.

Briefly, anesthetised mice were placed in a supine position, with the head supported at a 45° angle to the body. 14 nmol of NPS in 7 μL of ACSF or ACSF alone were applied alternatingly to each nostril; after 5 min, the procedure was repeated. Mice were then allowed to rest for 2 h before slice preparation and electrophysiological recording.

### 13.7 Voltage-Sensitive Dye Imaging (VSDI)

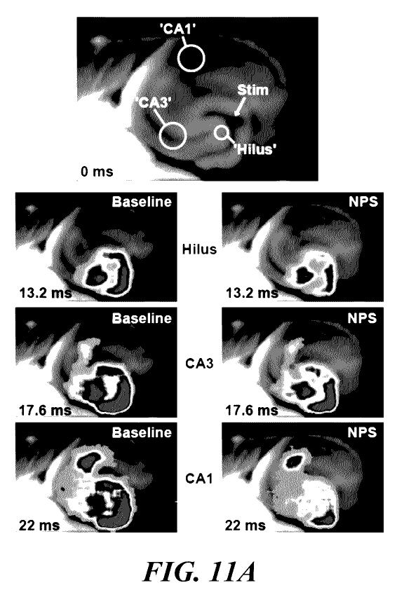

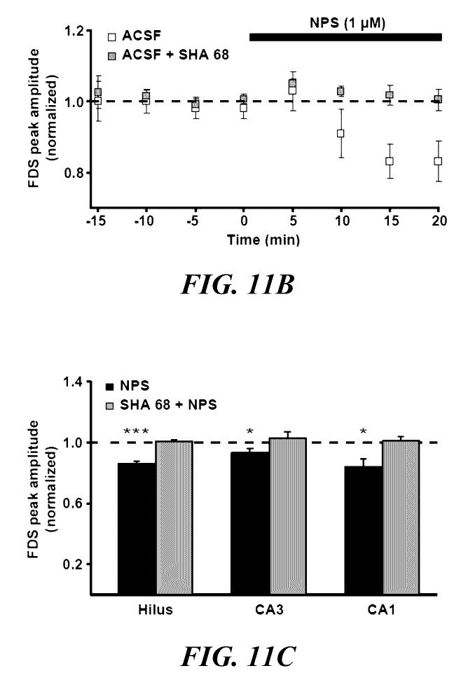

According to Maggio and Segal (2007) and Fanselow and Dong (2010), VSDI experiments were conducted in the VH. Horizontal brain slices (350 μm-thick) were prepared as described in Refojo et al., 2011 and/or von Wolff et al., 2011). Only the first two slices from the ventral surface of the brain in which the CA1 region was clearly visible were used for the measurements. Staining of slices with the voltage-sensitive dye Di-4-ANEPPS and VSDI were carried out at room temperature (23-25° C.). For staining, slices were kept for 15 min in carbogenated (95% O₂/5% CO₂) ACSF containing Di-4-ANEPPS (7.5 μg/mL; <0.1% DMSO). The ACSF (pH 7.4) consisted of (in mM): 125 NaCl, 2.5 KCl, 25 NaHCO₃, 1.25 NaH₂PO₄, 2 CaCl₂, 1 MgCl₂, and 25 glucose. Afterwards, slices were stored for at least 30 min in pure carbogenated ACSF. In the recording chamber, slices were continuously superfused with carbogenated ACSF (3 mL/min flow rate). VSDI and data analysis were performed using the MiCAM02 hard- and software package (BrainVision, Tokyo, Japan). The tandem-lens fluorescence microscope was equipped with the MiCAM02-HR camera and the 2× and 1× lens at the objective and condensing side, respectively. Acquisition settings were: 88×60 pixels frame size, 36.4×40.0 μm pixel size, and 2.2 ms sampling time. To reduce noise, four acquisitions subsequently recorded at intervals of 5 s were averaged. Neuronal activity was evoked by square pulse electrical stimuli (200 μs, 15-20 V) delivered to the dentate gyrus granule cell layer via a custom-made monopolar tungsten electrode (Teflon-insulated to the tip of 75 μm diameter). From recorded signals, the fractional change in fluorescence (ΔF/F) was calculated. For all quantifications, ΔF/F values were spatially and temporally smoothed using a 3×3×3 average filter. VSDI signals presented in images were smoothed with a 5×5×3 average filter. Pixelation of images was reduced by the interpolation function of the MiCAM02 software. For analysis of neuronal population activity in hippocampal subregions, three standardised circular regions of interest (ROIs) were manually set according to anatomical landmarks (FIG. 11A). The first ROI (r=3 pixels), named ‘Hilus’, was placed centrally into the hilus of the dentate gyrus, between the tip of the stimulation electrode and the proximal end of the CA stratum pyramidale. The second ROI ‘CA3’ (r=6 pixels) was positioned into the CA3 region near the dentate gyrus, but not overlapping with it. The third ROI ‘CA1’ (r=6 pixels) was placed into the CA1 subfield with a distance of approximately 400 μm from the visually identified distal end of the CA3 region. Both the ‘CA3’ and the ‘CA1’ ROI spanned the stratum oriens, stratum pyramidale, and stratum radiatum (lucidum). The average of smoothed ΔF/F values within a particular ROI served as final measure of neuronal population activity.

### 13.8 Electrophysiology

Brains were dissected and placed in ice-cold carbogenated ACSF. 350 μm horizontal slices containing the VH were prepared using a vibroslicer. Afterwards, the slices were incubated for 30 min at 34° C. and subsequently stored at room temperature. For experiments, slices were placed in a submerged recording chamber and continuously superfused with carbogenated ACSF at a flow rate of 3 mL/min. Square pulse electrical stimuli (50 μs pulse width) were delivered via a bipolar tungsten electrode (insulated to the tip, 50 μm pole diameter) that was positioned within the stratum radiatum of the CA1 region. All recordings were performed at room temperature, low-pass filtered at 1 kHz, and digitised at 5 kHz. Evoked field excitatory postsynaptic potentials (fEPSPs) were recorded using glass micro-electrodes (1 MΩ open-tip resistance), filled with ACSF and also positioned within the CA1 stratum radiatum. Stimulus intensities were adjusted in a manner to produce a fEPSP of ˜50% of that amplitude at which a population spike becomes clearly observable. Paired-pulse facilitation was measured at interstimulus intervals of 25, 50, 100, 200, and 400 ms and the paired-pulse ratio was calculated as fEPSP2 amplitude/fEPSP1 amplitude.

### 13.9 Statistics

Statistical analysis was performed using Sigma Stat 3.5 and GraphPad Prism 5.03. Statistical significance was assessed by means of the two-tailed unpaired Student's t-test, except for the VSDI experiments for which the two-tailed paired Student's t-test was used. Data are given as mean±SEM. In all graphs p values are depicted as follows: *p<0.05, **p<0.01, ***p<0.001.

13.10 Microinjections of NPS into the VH Reduce Anxiety in Mice

Before examining whether microinjections of NPS into the CA1 subfield of the VH modulate anxiety in adult C57BL/6N mice, the spread of the injected NPS using a fluorescent conjugate, i.e. Cy3-NPS was analysed. 30 min after injection, Cy3-NPS remained locally restricted to the VH and accumulated in single cells of the hippocampal pyramidal, radiate, and oriens layers (red fluorescence in FIG. 10A). An uptake of Cy3-NPS in nuclei of the amygdala was not observed (FIG. 10A). Thereupon, it was investigated whether unlabeled NPS produces similar anxiolytic effects as seen after intra-amygdalar (Jungling et al., 2008) and ICV injections (Xu et al., 2004; Jungling et al., 2008; Leonard et al., 2008; Rizzi et al., 2008), as well as after intranasal administration (see above). Standardised paradigms were employed to study anxiety-related behaviour and, for control, examined basal locomotion in the open field and anxiety- and locomotion-related parameters in both the dark-light test and the EPM. NPS did not affect locomotion in any of the three tests (FIG. 10B). 30 min after injection, NPS elicited a significant anxiolytic effect on the EPM, as evident from an increase in the percentage of time spent on the open arms (FIG. 10B: 28% increase compared to vehicle-treated mice). These results are in accordance with the examples above showing that intranasally applied NPS causes the strongest anxiolytic effect on the EPM.

### 13.11 Intranasally Applied NPS Impacts on Basal Neurotransmission and Plasticity at CA3-CA1 Synapses of the VH

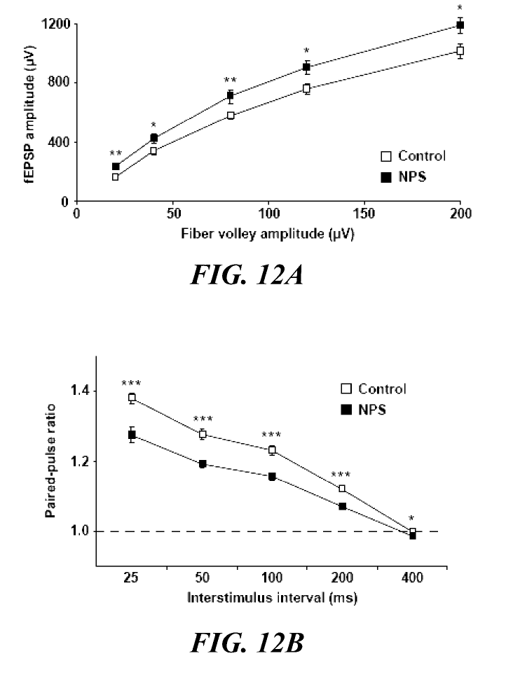

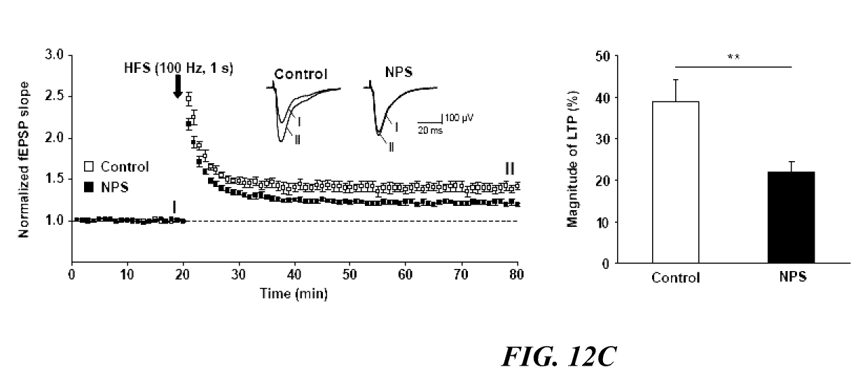

It is demonstrated above that bath application of NPS (1 μM) to VH slices from C57BL/6N mice decreases paired-pulse facilitation and long-term potentiation (LTP) at CA3-CA1 synapses via activation of NPSR (see above). To test whether these functional alterations also occur after intranasal administration of NPS, field potential recordings in VH slices from such treated animals and control mice were performed. Since the anxiolytic effect of intranasally applied NPS appeared after 30 min and lasted up to 4 h (see above), the electrophysiological measurements were conducted approximately 3 h after NPS or vehicle treatment. As shown in FIGS. 12A-12C, intranasal administration of NPS was followed by weakened paired-pulse facilitation and LTP at CA3-CA1 synapses. Additionally, input-output relationships at these synapses were studied. Consistent with an increased probability of transmitter release as suggested by the reduced paired-pulse ratio, intranasal NPS application led to a shift of input-output curves towards bigger fEPSP amplitudes (FIG. 12A).

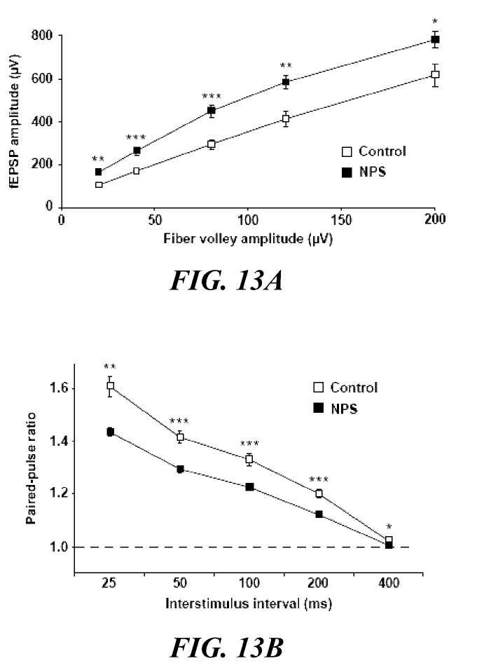

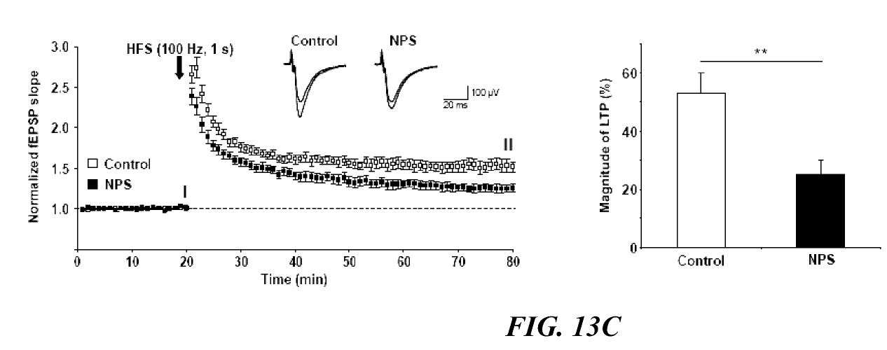

Next, it was examined whether these functional alterations also become manifest in mice displaying pathologically enhanced anxiety. For this purpose, the above described experiments were repeated in high-anxiety behaviour (HAB) mice (Kromer et al., 2005; Landgraf et al., 2007). Again, these measurements revealed a reduction in paired-pulse facilitation and LTP at CA3-CA1 synapses as well as an increase in the input-output relationship in VH slices from HAB animals that were treated intranasally with NPS (FIGS. 13A-13C).

13.12 NPS Weakens Neuronal Activity Flow from the Dentate Gyrus to Area CA1

Field potential recordings are a valuable tool to uncover changes in basal synaptic transmission and plasticity. However, they are not suited to unravel alterations in neuronal network dynamics, which might be a closer neurophysiological correlate of behaviour (Airan et al., 2007; Luo et al., 2008; Refojo et al., 2011). A high-speed voltage-sensitive dye imaging (VSDI) assay in mouse brain slices was established by the inventors enabling the investigation of several aspects of evoked neuronal activity flow from the dentate gyrus to area CA1 (Refojo et al., 2011; von Wolff et al., 2011). This activity flow is of high physiological relevance since the dentate gyrus represents a major input region and area CA1 an important output subfield of the hippocampus. By means of this VSDI assay, it was demonstrated that the anxiogenic neuropeptide corticotropin-releasing hormone (CRH) enhances this activity flow (Refojo et al., 2011; von Wolff et al., 2011). Here, similar experiments were conducted with NPS. As VSDI measure of neuronal activity, fast, depolarization-mediated imaging signals (‘FDSs’) were used. Stimulus-evoked FDSs in hippocampal slice preparations reflect action potentials and EPSPs (Airan et al., 2007; Refojo et al., 2011; von Wolff et al., 2011). Bath application of NPS (1 μM) to VH slices rapidly weakened the activity flow from the dentate gyrus to the CA1 subfield (FIG. 11A). This effect was completely abolished by the specific NPSR antagonist (R)-SHA 68 (10 μM) (FIG. 11B and FIG. 11C). NPS reduced the amplitude of FDSs in the dentate hilus, the CA3 region, and area CA1, indicating that NPS effects on neuronal activity in the VH are not limited to the CA1 subfield (FIG. 11A and FIG. 11C).

### 13.13 Summary

The experiments show that spatially restricted injections of NPS into the VH reduce anxiety in mice on the EPM. In addition, intranasal NPS application alters basal neurotransmission and plasticity at CA3-CA1 synapses of the VH, both in normal C57BL/6N and high-anxiety behaviour (HAB, CD1) mice (Kromer et al., 2005; Landgraf et al., 2007). These data are in conformity with (NPSR-mediated) functional changes upon bath application of NPS to VH slices (see above). Using high-speed membrane potential imaging, it has been additionally shown that NPS weakens evoked neuronal activity flow from the dentate gyrus to area CA1 in a NPSR-dependent manner. A thorough analysis of the VSDI experiments revealed that NPS does not only affect the functionality of the CA1 subfield (FIG. 11C) but that principal neurons of the CA3 region and the dentate gyrus also accumulate Cy3-NPS after ICV or intranasal administration (see above). Altogether, without being bound to this hypothesis it may be concluded that NPS impacts on the glutamatergic system of the VH and, thus, exerts in part its anxiolytic effects.

NPS activates presynaptic NPSRs at glutamatergic synapses in the amygdala, thereby causing an enhancement in the probability of transmitter release (Jungling et al., 2008). The data presented above showing reduced paired-pulse facilitation and a shift of input-output curves towards bigger fEPSP amplitudes, indicate that NPS leads to a similar effect at CA3-CA1 synapses of the VH. The observation of a decreased magnitude of LTP, also argues for an additional postsynaptic localisation of NPSRs on CA1 pyramidal neurons. Substantial support for this scenario is also given by the uptake of Cy3-NPS into these cells, both after its direct administration to the CA1 region (FIG. 10A) and after intranasal application (see above).

The mediation of anxiolysis of NPS via the above described neuromodulatory actions is probably due to the fact that CA1 pyramidal cells of the VH form excitatory synapses with amygdalar neurons (Andersen et al., 2007; Fanselow and Dong, 2010). The NPS-induced reduction of neuronal activity flow from the dentate gyrus to area CA1 might therefore result in a decreased activity of amygdalar anxiety circuits. Such a scenario appears at first glance contradictory to an enhancement in the probability of glutamate release at CA3-CA1 synapses. However, one has to take into account that CA3 pyramidal neurons typically respond with high-frequency (burst) spiking to suprathreshold depolarisations (Wong et al., 1979; Andersen et al., 2007). The resultant short-term facilitation of neurotransmission at CA3-CA1 synapses (as mimicked by the paired-pulse paradigm) is probably diminished to such a high degree in the presence of NPS that CA1 pyramidal cells exhibit reduced firing. In summary, the data presented above give experimental evidence for a direct involvement of the VH in NPS-induced anxiolysis. Moreover, it is shown that intranasally applied NPS has the capacity to profoundly modulate glutamatergic synaptic transmission and plasticity in the limbic system. VH appears to be an important brain structure for the regulation of fear and anxiety in mammals.

1-22. (canceled)

23. A method of treating an anxiety or sleep disorder, the method comprising nasally administering, to a subject in need thereof, a pharmaceutical composition comprising at least one peptide, wherein the peptide is an agonist of neuropeptide S receptor (NPSR).

24. The method of claim 23, wherein the anxiety disorder is a disorder selected from the group consisting of panic disorder with and without agoraphobia, phobia, posttraumatic stress disorder, generalised anxiety disorder, any other disease correlated with symptoms of pathological anxiety, and combinations thereof.

25. The method of claim 23, wherein the sleep disorder is a disorder selected from the group consisting of insomnia, hypersomnia, narcolepsy, idiopathic hypersomnia, excessive amounts of sleepiness, lack of alertness, lack of attentiveness, absentmindedness, lack of or aversion to movement or exercise, and combinations thereof.

26. The method of claim 23, wherein treatment of the sleep disorder comprises causing, promoting, or increasing arousal, awakening, alertness, activity, spontaneous movement, an anxiolytic effect, or a combination thereof in the subject.

27. The method of claim 23, wherein treatment of the anxiety disorder comprises relieving or healing avoidance anxiety, dissociative anxiety, vegetative symptoms related to anxiety symptoms, or a combination thereof in the subject.

28. The method of claim 24, wherein the phobia is selected from the group consisting of animal phobia, social phobia, height anxiety, claustrophobia, and agoraphobia.

29. The method of claim 27, wherein the dissociative anxiety is selected from the group consisting of flashbacks, depersonalisation, derealisation and intrusions.

30. The method of claim 27, wherein the vegetative symptoms related to anxiety symptoms arise from panic attacks.

31. The method of claim 23, wherein the pharmaceutical composition is selected from the group consisting of a nasal spray, nose drops, nose ointment, nose powder, and nose oil.

32. The method of claim 23, wherein the pharmaceutical composition further comprises a pharmaceutically acceptable compound, an enhancer, a bacterial component, a biological compound, a protein, another peptide, or a combination thereof.

33. The method of claim 23, wherein the peptide is present in the pharmaceutical composition at a therapeutically suitable concentration.

34. The method of claim 23, wherein after administration to the subject, the peptide is internalised with the receptor in a receptor-peptide-complex.

35. The method of claim 23, wherein the peptide is a non-naturally occurring peptide and contains one or more modifications selected from the group consisting of conservative substitutions, non-conservative substitutions, additions, and deletions.

36. The method of claim 23, wherein the peptide comprises the amino acid sequence Z¹ₘZ²ₙSFRNGVGX¹ᵢGX²ⱼKKTSFX³ₖRAKX⁴ₗZ²ₚZ³_{q};
wherein:
X¹ is a polar and/or neutral amino acid or a polar and/or neutral non-standard amino acid;
X² is a non-polar amino acid or a non-polar non-standard amino acid;
X³ is a polar and/or neutral amino acid or a basic amino acid or a polar and/or neutral non-standard amino acid or a basic non-standard amino acid;
X⁴ is a polar and/or neutral amino acid or a basic amino acid or a polar and/or neutral non-standard amino acid or a basic non-standard amino acid;
Z¹ is an N-terminal blocking group or —NH₂;
Z² is a member selected from the group consisting of a basic amino acid, a non-standard amino acid, a fluorescence tag, a hydrophobic tag, and a hydrophilic tag;
Z³ is a C-terminal blocking group or —COOH; and
i, j, k, l, m, n, p and q are integers independently selected from 0 to 25.

37. The method of claim 36, wherein X¹ is selected from the group consisting of tyrosine, threonine, glutamine, glycine, serine, cysteine, and asparagine.

38. The method of claim 36, wherein X² is selected from the group consisting of alanine, valine, methionine, leucine, isoleucine, proline, tryptophan, and phenylalanine.

39. The method of claim 36, wherein X³ is selected from the group consisting of tyrosine, threonine, glutamine, glycine, serine, cysteine, asparagine, lysine, arginine, and histidine.

40. The method of claim 36, wherein X⁴ is selected from the group consisting of tyrosine, threonine, glutamine, glycine, serine, cysteine, asparagine, lysine, arginine, and histidine.

41. The method of claim 36, wherein Z² is a basic amino acid selected from the group consisting of lysine, arginine, and histidine.

42. The method of claim 23, wherein the peptide comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, and 46, or a mutant or a fragment thereof.

43. The method of claim 23, wherein the peptide comprises an amino acid tag, an amino acid modification, or a combination thereof.

44. A pharmaceutical composition for nasal administration comprising at least one peptide, wherein the peptide is an agonist of neuropeptide S receptor (NPSR).
